SEQUENCE LISTING (8 sequences)

SEQ ID NO: 1 - protein, 8 aa, Artificial Sequence
  source: /note=“Description of Artificial Sequence Synthetic peptide”
  VARIANT (1)..(1): /replace=“Tyr” or “Asp” or “Ile” or “pGlu”
  VARIANT (5)..(5): /replace=“Abu”
  VARIANT (8)..(8): /replace=“Abu”
  SITE (1)..(8): /note=“Variant residues given in the sequence have no preference with respect to those in the annotations for variant positions”
KDRVAIHA

SEQ ID NO: 2 - protein, 8 aa, Artificial Sequence
  source: /note=“Description of Artificial Sequence Synthetic peptide”
  VARIANT (5)..(5): /replace=“Abu”
  VARIANT (8)..(8): /replace=“Abu”
  SITE (1)..(8): /note=“Variant residues given in the sequence have no preference with respect to those in the annotations for variant positions”
KDRVAIHA

SEQ ID NO: 3 - protein, 8 aa, Artificial Sequence
  source: /note=“Description of Artificial Sequence Synthetic peptide”
  VARIANT (5)..(5): /replace=“Abu”
  VARIANT (8)..(8): /replace=“Abu”
  SITE (1)..(8): /note=“Variant residues given in the sequence have no preference with respect to those in the annotations for variant positions”
DDRVAIHA

SEQ ID NO: 4 - protein, 8 aa, Artificial Sequence
  source: /note=“Description of Artificial Sequence Synthetic peptide”
  VARIANT (5)..(5): /replace=“Abu”
  VARIANT (8)..(8): /replace=“Abu”
  SITE (1)..(8): /note=“Variant residues given in the sequence have no preference with respect to those in the annotations for variant positions”
YDRVAIHA

SEQ ID NO: 5 - protein, 8 aa, Artificial Sequence
  source: /note=“Description of Artificial Sequence Synthetic peptide”
  VARIANT (5)..(5): /replace=“Abu”
  VARIANT (8)..(8): /replace=“Abu”
  SITE (1)..(8): /note=“Variant residues given in the sequence have no preference with respect to those in the annotations for variant positions”
IDRVAIHA

SEQ ID NO: 6 - protein, 8 aa, Artificial Sequence
  source: /note=“Description of Artificial Sequence Synthetic peptide”
  VARIANT (5)..(5): /replace=“Abu”
  VARIANT (8)..(8): /replace=“Abu”
  SITE (1)..(8): /note=“Variant residues given in the sequence have no preference with respect to those in the annotations for variant positions”
NDRVAIHA

SEQ ID NO: 7 - protein, 8 aa, Artificial Sequence
  source: /note=“Description of Artificial Sequence Synthetic peptide”
DRVYIHPF

SEQ ID NO: 8 - protein, 22 aa, Artificial Sequence
  source: /note=“Description of Artificial Sequence Synthetic peptide”
YGRKKRRQRRRLYRSPAMPENL

(19) United States
(12) Patent Application Publication
(10) Pub. No.: US 2022/0257698 A1
(43) Pub. Date: Aug. 18, 2022

(54) ANGIOTENSIN TYPE 2 (AT2) RECEPTOR AGONISTS FOR USE IN THE TREATMENT OF CANCER

(71) Applicant: LANTHIOPEP B.V., Groningen (NL)

(72) Inventors: Pawel Namsolleck, Groningen (NL); Anneke Kuipers, Groningen (NL); Louwe De Vries, Groningen (NL); Claudio Festuccia, Coppito (AQ) (IT)

(21) Appl. No.: 17/630,636

(22) PCT Filed: Aug. 3, 2020
(86) PCT No.: PCT/EP2020/071775

§ 371 Date: Jan. 27, 2022

(30) Foreign Application Priority Data
Aug. 2, 2019 (EP) 19189729.7

(51) Int. Cl. A61K 38/12; A61P 35/00; A61K 45/06

(52) U.S. Cl. CPC A61K 38/12; A61K 45/06; A61P 35/00

(57) ABSTRACT

The present invention relates to the cyclic peptides that are agonists of the angiotensin II type 2 receptor (hereinafter the AT2 receptor) useful in the treatment of different types of solid cancer, in particular brain, colon, lung and ovarian cancer. The invention further relates to pharmaceutical compositions containing them and uses thereof for the treatment of cancer.

### FIELD OF THE INVENTION

The present invention relates to the cyclic peptides that are agonists of the angiotensin II type 2 receptor (hereinafter the AT2 receptor) useful in the treatment of different types of cancer, in particular solid cancers. The invention further relates to pharmaceutical compositions containing them and uses thereof for the treatment of specific types of cancer.

Cyclic peptides useful in the methods and uses of the invention include cyclized peptide variants of Angiotensin(1-7) (Ang(1-7)), in particular thioether-bridged peptide variants of Ang(1-7), such as variants having an N-terminal extension with a single amino acid residue (abbreviated to “XcAng(1-7)”).

### BACKGROUND

The endogenous hormone angiotensin (abbreviated to “AngII”) is a linear octapeptide (Asp1-Arg2-Val3-Tyr4-Ile5-His6-Pro7-Phe8), and is the active component of the renin-angiotensin system (RAS). It is produced by the sequential processing of the pro-hormone angiotensinogen by renin and angiotensin converting enzyme (ACE). The RAS plays an important role in the regulation of blood pressure, body fluid and electrolyte homeostasis.

AngII exerts these physiological actions in many organs including the kidneys, the adrenal glands, the heart, blood vessels, the brain, the gastrointestinal tract and the reproductive organs (de Gasparo et al, Pharmacol. Rev. (2000) 52, 415-472).

Two main classes of AngII receptors have been identified, and designated as the type 1 receptor (hereinafter the AT1 receptor) and the AT2 receptor. The AT1 receptor is expressed in most organs, and is believed to be responsible for the majority of the biological effects of AngII. The AT2 receptor is more prevalent than the AT1 receptor in fetal tissues, the adult ovaries, the adrenal medulla and the pancreas. An equal distribution is reported in the brain and uterus (Ardaillou, J. Am. Soc. Nephrol., 10, S30-39 (1999)).

Several studies in adult individuals appear to demonstrate that, in the modulation of the response following AngII stimulation, activation of the AT2 receptor, has opposing effects to those mediated by the AT1 receptor. The AT2 receptor has also been shown to be involved in apoptosis and inhibition of cell proliferation (see de Gasparo et al, supra). Further, it seems to play a role in blood pressure control. The functional relevance of AT2 receptors in cardiovascular disease is discussed in Jones et al. (Pharmacology & Therapeutics 120 (2008) 292-316).

The expression of AT2 receptors has also been shown to increase during pathological circumstances, such as vascular injury, wound healing and heart failure (see de Gasparo et al, supra). The expected pharmacological effects of agonism of the AT2 receptor are described generally in de Gasparo et al, supra.

AT2 receptor agonists have been shown to be of potential utility in the treatment and/or prophylaxis of disorders of the alimentary tract, such as dyspepsia and irritable bowel syndrome, as well as multiple organ failure (see WO 99/43339).

Angiotensin AT1 receptor antagonists have been disclosed in inter alia European patent applications EP 409 332, EP 512 675; international patent applications WO 94/27597, WO 94/02142, WO 95/23792 and WO 94/03435; and U.S. Pat. Nos. 5,091,390, 5,177,074, 5,412,097, 5,250,521, 5,260,285, 5,376,666, 5,252,574, 5,312,820, 5,330,987, 5,166,206, 5,932,575 and 5,240,928.

US 2009/326026 discloses the use of tricyclic, imidazole-containing compounds as AT2 agonist. WO 04/046128 relates to bicyclic compounds, which are useful as selective agonists of the AT2 receptor.

Peptide and non-peptide AT2 receptor agonists, unrelated structurally to those described herein, and potential uses thereof, have been disclosed in, for example, international patent applications WO 00/38676, WO 00/56345, WO 00/09144, WO 99/58140, WO 99/52540, WO 99/46285, WO 99/45945, WO 99/42122, WO 99/40107, WO 99/40106, WO 99/39743, WO 99/26644, WO 98/33813, WO 00/02905 and WO 99/46285; U.S. Pat. No. 5,834,432; and Japanese patent application JP 143695.

Linear peptide variants of Angiotensin(1-7) with or without N-terminal extensions of 1 to 3 amino acids have been described by K. Rodgers et al., for example in WO99/40106, WO99/52540 and WO96/39164. US2004/176302 (WO2002087504) also relates to linear angiotensinogen, angiotensin I, angiotensin II, AT2 receptor agonists for inhibiting tumor cell proliferation in vitro. None of these disclosures shows or suggests the advantageous properties of the cyclic thioether-bridged peptide variants of the present application.

Thioether-bridged peptide variants of Angiotensin(1-7) are also known in the art. See for example Kluskens et al. (J Pharmacol Exp Ther. 2009 March; 328(3):849-54), WO 08/130217 and WO 12/070936.

The functional and clinical relevance of the AT2 receptor in cancer has been discussed in the art. For instance, AT2 receptor underexpression could be shown in human breast cancer samples (Tovart H et al. Comput Biol Chem. 2015 Aug. 22) and negative correlation of AT2 receptor expression with colorectal carcinoma progression was observed in human patients (Zhou L et al. Pathobiology; 2014; 81(4): 169-75). The tumor growth suppressing properties of the AT2 receptor was further confirmed for murine rectal cancer cells by in vitro AT2 receptor knock-down studies (Zhou L et al. Pathobiology; 2014; 81(4): 169-75).

Vinson G P et al, Endocr Relat Cancer. 2012 Feb. 13; 19(1) reviews the ambiguous role of the renin-angiotensin system in breast cancer. A potential therapeutic use of ACE inhibitors and AT1 receptor blockers is discussed and it is speculated about a role for AT2 receptor agonists in breast cancer, though this awaits full investigation. This review focuses on AT1 receptor antagonism with antibodies and is completely silent on cyclic peptide variants as AT2 agonists.

AT2 receptor overexpression and AT2 receptor stimulation as a therapeutic option for the treatment of cancer have been also discussed in the art.

Recombinant AT2 receptor overexpression in human prostate and bladder cancer cells resulted in tumor progression inhibition in vivo. (Pei N et al. J Exp Clin Cancer Res. 2017 Jun. 9; 36(1):77); Li J, J Cancer. 2016 Jan. 1; 7(2):184-91). Furthermore, recombinant overexpression of the AT2 receptor in human Lewis lung carcinoma cells reduced number of tumor nodules in mice lungs.

Intracellular AT2 receptor stimulation with an AT2 receptor agonist was also shown to induce rapid cell death in quiescent human leiomyosarcoma cells in vitro (Zhao Y et al. J. Clin Sci (Lond). 2015; 128: 567-578) and also reduced liver metastases of mouse colorectal cancer cells in vivo (Ager El et al. Cancer Cell Int. 2010 Jun. 28; 10:19).

The combination of AT2 receptor overexpression in conjunction with the treatment with an AT1 receptor antagonist synergistically reduced tumor volume in a human lung adenocarcinoma cells in vivo model (Su Y, Biomaterials. 2017 September; 139:75-90) whereas the combination of AT2 receptor overexpression with an AT2 receptor agonist resulted in reduced tumor weight and promoted apoptosis in a pancreatic ductal adenocarcinoma in vivo model (Ishiguro S et al. Cancer Biol Ther. 2015; 16(2):307-16). The combination of an AT2 receptor agonist and an AT1 receptor antagonist synergistically inhibited growth of epithelial ovarian carcinoma in vivo (Park Y A, Gynecol Oncol. 2014 October; 135(1):108-17).

Natural linear angiotensin(1-7), acting via the Mas receptor, has been reported to have antitumor activity (Mao Y et al 2018, Int. J. Biol. Sci. 14, 57-68; Hinsley E E et al 2017, Eur J Oral Sci 125 247-257; Cambados N et al 2017 Oncotarget 8, 88475-88487; Chen et al 2017 Oncotarget 8, 354-363; Pei et al 2016 Molecular Cancer Therapeutics 15, 37-47; Liu et al 2015, Mol Med 21, 626-36; Krishnan et al 2013 Prostate. 73, 71-82; Soto-Pantoja et al 2009 Mol Cancer Ther 8, 1676-1683; Menon et al 2007, Cancer Res. 67, 2809-15. In addition Angiotensin-(1-7) has been stabilized by a non-natural cyclic amino acid retaining the capacity to inhibit tumor cells (Wester A et al 2017 Amino Acids. October; 49(10):1733-1742).

US 2014/296143 discloses the use of the natural linear angiotensin-(1-7) peptide (Asp-Arg-Val-Tyr-Ile-His-Pro) as an anti-cancer and chemoprevention therapeutic for lung and breast tumors.

However, thioether-bridged peptide variants of Angiotensin(1-7) with an N-terminal extension being useful in the treatment of brain, colon, lung or ovarian cancer have not been taught in the art.

A brain tumor develops when abnormal cells form within the brain. Cancerous brain tumors can be divided into primary tumors, which start within the brain, and secondary tumors, which have spread from other organs, known as brain metastasis tumors. Glioblastoma multiforme (GBM) is the most common (50.4%) and aggressive malignant adult primary brain tumor, as it is highly invasive and proliferative, and resistant to standard therapeutic strategies. Current treatments for malignant glioma include a combination of surgical resection, radiotherapy or radio-surgery, and chemotherapy (alkylating agents such as typically temozolomide). GBM is associated with the presence of cancer stem cells (CSCs) possessing the ability for perpetual self-renewal and proliferation and producing downstream progenitor cells that drive tumor growth. Other primary brain tumors comprise meningiomas (20.8%), pituitary adenomas (15%) and nerve sheath tumors (8%) (Park B J, et al. (2009). “Epidemiology”. In Lee J H (ed.). Meningiomas: Diagnosis, Treatment, and Outcome. Springer).

Colon cancer, also known as bowel cancer or colorectal cancer (CRC) is the development of cancer from the colon or rectum. It is the third most common type of cancer, making up about 10% of all cases worldwide. Treatments used include surgery, radiation therapy, chemotherapy and targeted therapy and combinations thereof. Cancers that are confined within the wall of the colon may be curable with surgery, while cancer that has spread widely is usually not curable, with management being directed towards improving quality of life and symptoms.

Lung cancer is one of the most common and serious types of cancer. Cancer that begins in the lungs is called primary lung cancer. Cancer that spreads to the lungs from another organ is known as secondary lung cancer. There are two main forms of primary lung cancer, i.e. (i) non-small-cell lung cancer—the most common form, accounting for more than 87% of cases. It can be one of three types: squamous cell carcinoma, adenocarcinoma or large-cell carcinoma. (ii) small-cell lung cancer—a less common form that usually spreads faster than non-small-cell lung cancer. If the tumor is diagnosed early, surgery to remove cancerous cells confined to a small area may be applied. Otherwise, radiotherapy, chemotherapy or targeted therapies may be used.

Ovarian cancer is a tumor that forms in or on an ovary. It results in abnormal cells that metastasize to other organs. Ovarian cancer types comprise: (i) epithelial tumors, in the tissue that covers the outside of the ovaries. About 90 percent of ovarian cancers are epithelial tumors, (ii) stromal tumors, in the ovarian tissue that contains hormone-producing cells. About 7 percent of ovarian tumors are of stromal origin, (iii) germ cell tumors, in the egg-producing cells. Germ cell tumors are rare. Treatment of ovarian cancer usually involves surgery, chemotherapy, and sometimes radiotherapy, regardless of the subtype of ovarian cancer. Despite the fact that 60% of ovarian tumors have estrogen receptors, ovarian cancer is only rarely responsive to hormonal treatment.

There is still a huge medical need for providing alternative options for treating these and other types of cancer, for alleviating symptoms and improving patient's life.

### SUMMARY OF THE INVENTION

The inventors of the present invention have surprisingly found that thioether-bridged peptide variants of Ang(1-7), in particular thioether-bridged peptide variants of Ang(1-7), extended with an additional amino acid at the N-terminus are useful in the treatment of cancer, in particular in the treatment by AT2 receptor agonist, XcAng(1-7), of solid cancers, such as brain, colon, ovarian or lung cancer.

Accordingly, the present disclosure provides thioether-bridged Ang(1-7) peptides extended with an additional amino acid at the N-terminus (abbreviated “XcAng(1-7)”) for use in the treatment of cancer.

In certain embodiments, the XcAng(1-7) according to the present disclosure is for use in the treatment of a solid cancer. In certain embodiments, the XcAng(1-7) according to the present disclosure is for use in the treatment of a brain cancer, a colon cancer, a lung cancer, and/or an ovarian cancer. In preferred embodiments, the XcAng(1-7) is for use in the treatment of a colon cancer. In another preferred embodiment, the XcAng(1-7) is for use in the treatment of a brain cancer. In a particular embodiment, the brain cancer is glioblastoma multiforme.

In certain embodiments, said thioether-bridged peptide variant of XAng(1-7) refers to a cyclic peptide.

In certain aspects, the present disclosure provides a cyclic peptide consisting of the amino acid sequence

(SEQ ID NO: 1)
Xaa1-Asp-Arg-Val-Abu/Ala-Ile-His-Abu/Ala

comprising a thioether-bridge linkage between the side chains of Abu/Ala at position 5 and Abu/Ala at position 8, wherein Xaa1 is selected from the group consisting of Lys, Tyr, Asp, pGlu and lle, for use in the treatment of cancer.

In an embodiment, the cyclic peptide according to the present disclosure is for use in the treatment of cancer.

In a further embodiment, the cyclic peptide according to the present disclosure is for use in the treatment of cancer, wherein said cancer is a solid cancer.

In an embodiment, said solid cancer is a brain cancer, colon cancer, lung cancer, and/or ovarian cancer.

In an embodiment, the present disclosure provides a cyclic peptide according to the present disclosure for use in the treatment colon cancer.

In another embodiment, the present disclosure provides a cyclic peptide according to the present disclosure for use in the treatment of brain cancer, in particular glioblastoma.

In an embodiment, the present disclosure provides a cyclic peptide according to the present disclosure for the use according to the present disclosure, wherein Xaa1 of said cyclic peptide is a D-stereoisomer.

In an embodiment, the present disclosure provides a cyclic peptide according to the present disclosure for the use according to the present disclosure, wherein Xaa1 of said cyclic peptide is a D-stereoisomer of Lys.

In an embodiment, the present disclosure provides a cyclic peptide according to the present disclosure for the use according to the present disclosure, wherein position 5 of said cyclic peptide is a D-stereoisomer of Ala.

In an embodiment, the present disclosure provides a cyclic peptide according to the present disclosure for the use according to the present disclosure, wherein position 8 of said cyclic peptide is an L-stereoisomer of Ala.

In an embodiment, the present disclosure provides a cyclic peptide according to the present disclosure for the use according to the present disclosure, wherein position 8 of said cyclic peptide is an L-stereoisomer of Ala and wherein position 1 is a D-stereoisomer of Lys.

In an embodiment, the present disclosure provides a cyclic peptide according to the present disclosure for the use according to the present disclosure, wherein position 5 of said cyclic peptide is a D-stereoisomer of Ala and position 8 is an L-stereoisomer of Ala.

In an embodiment, the present disclosure provides a cyclic peptide according to the present disclosure for the use according to the present disclosure, having an amino acid sequence of Lys-Asp-Arg-Val-Abu/Ala-Ile-His-Abu/Ala (SEQ ID NO: 2) under the provision that the peptide does not contain two Abu (2-aminobutyric acid) residues.

In an embodiment of the disclosure, the cyclic peptide for use in the treatment of cancer has an amino acid sequence selected from the group consisting of:

(SEQ ID NO: 2)
Lys-Asp-Arg-Val-Abu/Ala-Ile-His-Abu/Ala
(abbreviated to “K-cAng(1-7)”)
(SEQ ID NO: 3)
Asp-Asp-Arg-Val-Abu/Ala-Ile-His-Abu/Ala
(abbreviated to “D-cAng(1-7)”)
(SEQ ID NO: 4)
Tyr-Asp-Arg-Val-Abu/Ala-Ile-His-Abu/Ala
(abbreviated to “Y-cAng(1-7)”)
(SEQ ID NO: 5)
Ile-Asp-Arg-Val-Abu/Ala-Ile-His-Abu/Ala
(abbreviated to “I-cAng(1-7)”)
or
(SEQ ID NO: 6)
Asn-Asp-Arg-Val-Abu/Ala-Ile-His-Abu/Ala
(abbreviated to “N-cAng(1-7)”)

with a thioether-bridge between the side chains of Abu/Ala at position 5 and Abu/Ala at position 8, and with the provision that the peptide does not contain two Abu (2-aminobutyric acid) residues.

In an embodiment of the disclosure, the cyclic peptide for use in the treatment of cancer is selected from the group consisting of:

(SEQ ID NO: 2)
Lys-Asp-Arg-Val-Abu/Ala-Ile-His-Abu/Ala

with a thioether-bridge between the side chains of Abu/Ala at position 5 and Abu/Ala at position 8 and with the provision that the peptide does not contain two Abu (2-aminobutyric acid) residues.

In an embodiment of the present disclosure, the cyclic peptide is a peptide compound disclosed in WO2012/070936.

In an embodiment, said use in the treatment of cancer is the use in the treatment of solid cancer, preferably for use in the treatment of brain cancer, colon cancer, lung cancer, and/or ovarian cancer.

In an embodiment, the present disclosure provides a pharmaceutical composition comprising a cyclic peptide according to the present disclosure for use in the treatment of cancer. In another embodiment, the present disclosure provides a pharmaceutical composition comprising a cyclic peptide according to the present disclosure and a pharmaceutically acceptable adjuvant, diluent or carrier for use in the treatment of cancer.

In an embodiment, said pharmaceutical composition comprising a cyclic peptide according to the present disclosure is for use in the treatment of a solid cancer.

In an embodiment, said pharmaceutical composition comprising a cyclic peptide according to the present disclosure is for use in the treatment of brain cancer, colon cancer, lung cancer or ovarian cancer.

The cyclic peptides according to the present disclosure suited for use in the treatment of cancer have the advantage that they bind selectively to, and exhibit agonist activity at the AT2 receptor.

The cyclic peptides according to the present disclosure for use in the treatment of cancer may also have the advantage to be more efficacious, be less toxic, be longer acting, be more potent, produce fewer side effects, be more easily absorbed, and/or have a better pharmacokinetic profile (e.g. higher oral bioavailability and/or lower clearance) than the compounds known in the prior art.

### BRIEF DESCRIPTION OF THE DRAWINGS

FIG. 1: A-D In vivo efficacy of KcAng(1-7) against 4 different colon cancer PDXs in a murine model. Mice were treated either with vehicle (open circle), treated with 0.2 μg/kg/d KcAng(1-7) (filled square), or treated with 30 μg/kg/d KcAng(1-7) (filled triangle).

FIG. 2: In vivo efficacy of KcAng(1-7) against the HN10309 head and neck PDX model in mice. Animals were treated either with vehicle (open circle), treated with 0.2 μg/kg/d KcAng(1-7) (filled square), or treated with 30 μg/kg/d KcAng(1-7) (filled triangle).

FIG. 3: In vivo efficacy of KcAng(1-7) against the Lu7433 lung cancer PDX model in mice. Animals were treated either with vehicle (open circle), treated with 0.2 μg/kg/d KcAng(1-7) (filled square), or treated with 30 μg/kg/d KcAng(1-7) (filled triangle).

FIG. 4: In vivo efficacy of KcAng(1-7) against the MaCa4151 breast cancer PDX model in mice. Animals were treated either with vehicle (open circle), treated with 0.2 μg/kg/d KcAng(1-7) (filled square), or treated with 30 μg/kg/d KcAng(1-7) (filled triangle).

FIG. 5: In vivo efficacy of KcAng(1-7) against the OvCa 13329 ovarian cancer PDX model in mice. Animals were treated either with vehicle (open circle), treated with 0.2 μg/kg/d KcAng(1-7) (filled square), or treated with 30 μg/kg/d KcAng(1-7) (filled triangle).

FIG. 6: A-D Histology of extracted tumor tissues from colon cancer PDX model after treatment with KcAng(1-7) or vehicle. Results from immunohistochemistry analyses of cleaved caspase 3 (CC3, apoptosis), Ki67 (proliferation) in Co9689A PDX.

FIG. 7: A-D Tumor RNA expression levels of ATIP (A), TP53 (B), TIMP1 (C) and Bax (D) in relation to healthy human colon tissue. Colon tumor tissues were extracted from mouse Co7809 model treated with KcAng(1-7) or vehicle.

FIG. 8: In vivo efficacy of KcAng(1-7) against the U87MG glioblastoma CDX model in mice. Animals were treated either with vehicle (open circle), treated with 0.2 μg/kg/d KcAng(1-7) (filled square), treated with 16 mg/kg temozolomide (TMZ) (filled circles) or treated with a combination of KcAng(1-7) and TMZ (filled triangle).

FIG. 9: Time of tumor progression—TTP (days) in U87MG CDX treated either with vehicle (open circle), treated with 0.2 μg/kg/d KcAng(1-7) (filled square), treated with 16 mg/kg temozolomide (TMZ)(filled circles) or treated with a combination of KcAng(1-7) and TMZ (filled triangle).

FIG. 10: In vivo efficacy of KcAng(1-7) against the U87MG glioblastoma CDX model in mice. Animals were treated either with vehicle (open circle), treated with 20 mg/kg/d Losartan (filled squares), treated with 1 μg/kg/d KcAng(1-7) (filled circles), or treated with a combination of KcAng(1-7) and Losartan (open triangle).

FIG. 11: Time of tumor progression—TTP (days) in U87MG CDX treated either with vehicle (open circle), treated with 20 mg/kg/d Losartan (filled squares), treated with 1 μg/kg/d KcAng(1-7) (filled circles), or treated with a combination of KcAng(1-7) and Losartan (open triangle).

FIG. 12: Tumor hemoglobin level in U87MG CDX treated either with vehicle (open circle), treated with 1 μg/kg/d KcAng(1-7) (filled squares), treated with 20 mg/kg/d Losartan (filled circles), or treated with a combination of KcAng(1-7) and Losartan (open triangle).

FIG. 13: In vivo efficacy of KcAng(1-7) against the U251MG glioblastoma CDX model in mice. Animals were treated either with vehicle (open circle), treated with 20 mg/kg/d Losartan (filled squares), treated with 1 μg/kg/d KcAng(1-7) (filled circles), or treated with a combination of KcAng(1-7) and Losartan (open triangle).

FIG. 14: Time of tumor progression—TTP (days) in U251MG CDX treated either with vehicle (open circle), treated with 20 mg/kg/d Losartan (filled squares), treated with 1 μg/kg/d KcAng(1-7) (filled circles), or treated with a combination of KcAng(1-7) and Losartan (open triangle).

FIG. 15: Tumor hemoglobin level in U251MG CDX treated either with vehicle (open circle), treated with 20 mg/kg/d Losartan (filled squares), treated with 1 μg/kg/d KcAng(1-7) (filled circles), or treated with a combination of KcAng(1-7) and Losartan (open triangle).

### DETAILED DESCRIPTION OF THE INVENTION

### Definitions

The term “variant” as used herein refers to a peptide that possesses in its sequence at least 50% of the amino acid residues of another peptide and mimics the function or action of the other peptide.

The term “peptide” means a molecule having less than or equal to 50 amino acids.

The term “cyclic peptide” refers to a stretch of amino acids, a peptide or a polypeptide having a secondary structure formed by one or more intramolecular bonds. Not the entire stretch of amino acids or peptide or polypeptide needs to be circular. In an embodiment of the present disclosure a cyclic peptide is a monocyclic peptide. In another embodiment a cyclic peptide comprises peptides such as naturally occurring or artificial peptides, as well as peptides that are fragments or domains of whole proteins. In a further embodiment, a cyclic peptide is an amidated cyclic peptide.

The terms “thioether” or “thioether-bridge” refer to a sulfur atom bonded to two different carbon or hetero atoms in a respective molecule. In one embodiment, the thioether-bridge is formed after post-translational dehydration of one or more serine or threonine residues and coupling of said dehydrated residues to a cysteine. In another embodiment, the thioether-bridged peptide is formed by base-assisted sulfur extrusion of a disulfide-bridged peptide. In one embodiment, the thioether-bridge is part of a lanthionine (Ala-S-Ala) or a methyllanthionine (Abu-S-Ala or Ala-S-Abu). Lanthionine is a non-proteinogenic amino acid with the chemical formula (HOOC—CH(NH₂)—CH₂—S—CH₂—CH(NH₂)—COOH), composed of two alanine residues that are crosslinked on their β-carbon atoms by a thioether-bridge. Methyllanthionine is a non-proteinogenic amino acid with the chemical formula (HOOC—CH(NH₂)—CH(CH₃)—S—CH₂—CH(NH₂)—COOH).

The term “dehydrated residue” refers to a modified amino acid residue that underwent a chemical reaction, which involved the loss of a water molecule from the reacting molecule. In one embodiment, the “dehydrated residue” is a dehydrated serine or a dehydrated threonine.

The term “N-terminus” of a given polypeptide sequence is a contiguous length of the given polypeptide sequence that begins at or near the N-terminal residue of the given polypeptide sequence or it is a terminal pyroglutamate (pGlu).

As used herein, the terms “treat”, “treating”, or the like, mean to alleviate symptoms, eliminate the causation of symptoms, either on a temporary or permanent basis, or to prevent or slow the appearance of symptoms of the named disorder or condition.

“Preventing” or “prevention” refers to a reduction in risk of acquiring or developing a disease (i.e. causing at least one of the clinical symptoms of the disease not to develop in a subject that may be exposed to a disease-causing agent, or predisposed to the disease in advance of disease onset). “Prevention” also refers to methods which aim to prevent the onset of a disease or its symptoms or which delay the onset of a disease or its symptoms.

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

“Subject” or “species” or “individual” as used in this context refers to any mammal, including rodents, such as mouse or rat, and primates, such as cynomolgus monkey (Macaca fascicularis), rhesus monkey (Macaca mulatta) or humans (Homo sapiens). Preferably, the subject is a primate, most preferably a human.

A cyclic peptide which “binds selectively” to the AT2 receptor, has an affinity ratio for the relevant compound (AT2:AT1) which is at least 5:1, preferably at least 10:1 and more preferably at least 20:1.

The terms “inhibition” or “inhibit” or “reduction” or “reduce” or “neutralization” or “neutralize” refer to a decrease or cessation of any phenotypic characteristic (such as binding or a biological activity or function) or to the decrease or cessation in the incidence, degree, or likelihood of that characteristic. The “inhibition”, “reduction” or “neutralization” needs not to be complete as long as it is detectable using an appropriate assay. In some embodiments, by “reduction” or “inhibition” or “neutralization” is meant the ability to cause a decrease of 20% or greater. In another embodiment, by “reduction” or “inhibition” or “neutralization” is meant the ability to cause a decrease of 50% or greater. In yet another embodiment, by “reduction” or “inhibition” or “neutralization” is meant the ability to cause an overall decrease of 75%, 85%, 90%, 95%, or greater.

The terms “increase” or “increasing” or “enhance” or “enhancing” refer to an increase of any phenotypic characteristic (such as binding, a biological activity or function) or to the increase in the incidence, degree, or likelihood of that characteristic. The “increase” or “enhancing” needs not to be the maximum effect as long as it is detectable using an appropriate assay. In some embodiments, by “increasing” or “enhancing” is meant the ability to cause an increase of 20% or greater. In another embodiment, by “increasing” or “enhancing” is meant the ability to cause an increase of 50% or greater. In yet another embodiment, by “increasing” or “enhancing” is meant the ability to cause an overall increase of 75%, 85%, 90%, 95%, or greater.

The term “antagonist” as used herein refers to a molecule that interacts with an antigen and inhibits a biological activity or function or any other phenotypic characteristic of an antigen.

The term “agonistic” as used herein refers to a molecule that interacts with an antigen and increases or enhances a biological activity or function or any other phenotypic characteristic of the target antigen and/or upregulates the expression of the antigen.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Compositions” of the present disclosure may be used for therapeutic or prophylactic applications. The present disclosure, therefore, includes a pharmaceutical composition containing a cyclic peptide as disclosed herein and a pharmaceutically acceptable carrier or excipient.

The term “Pharmaceutical composition” refers to a preparation which is in such form as to permit the biological activity of an active ingredient contained therein to be effective, and which contains no additional components which are unacceptably toxic to a subject to which the formulation would be administered.

The term “Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

A “therapeutically effective amount” or “effective amount”, as used herein, refers to the amount of a cyclic peptide according to the present disclosure, to elicit the desired physiological change in the cell or tissue to which it is administered.

As used herein, amino acid residues will be indicated either by their full name or according to the standard three-letter or one-letter amino acid code. “Natural occurring amino acids” means the following amino acids:

TABLE 1
Natural occurring amino acids

| | Three letter | One letter |
|---|---|---|
| Amino acid | code | code |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “Abu” refers to α-Aminobutyric acid or 2-aminobutyric acid, which is a non-proteinogenic alpha-amino acid with chemical formula C₄H₉NO₂.

The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

### EMBODIMENTS

The XcAng(1-7) according to the present disclosure may be used in therapeutic methods.

The inventors of the present invention have surprisingly found that peptide variants of Angiotensin(1-7), in particular thioether-bridged peptide peptide variants of Ang(1-7), in more particular thioether-bridged peptide variants of Ang(1-7) extended with an additional single amino acid at the N-terminus are useful in the treatment of cancer, in particular in the treatment of solid cancers, such as brain, colon, ovarian or lung cancer.

According to a further aspect of the present disclosure, the present disclosure provides a XcAng(1-7) according to the present disclosure for use in the treatment of cancer, in which endogenous production of AT2 receptor agonists is deficient, and/or a cancer where an increase in the effect of AT2 receptor agonists is desired or required, and/or a cancer where AT2 receptors are expressed and their stimulation is desired or required, and/or a cancer wherein ligand-mediated upregulation of the AT2 receptor expression is desired.

In an embodiment, said use in the treatment of cancer comprises administration of a therapeutically effective amount of a XcAng(1-7) according to the present disclosure to a subject suffering from, or susceptible to such a cancer.

Accordingly, in an embodiment, the present disclosure provides a peptide variant of XcAng(1-7) for use in the treatment of cancer.

In an embodiment, said peptide variant of XcAng(1-7) for use in the treatment of cancer is a peptide. In another embodiment, said variant is a cyclic peptide. In a further embodiment, said cyclic peptide is a thioether-bridged cyclic peptide. In an embodiment, said thioether-bridged cyclic peptide is extended with an additional amino acid at the N-terminus.

In an embodiment, said additional single amino acid residue is a natural occurring amino acid. In an embodiment, said additional single amino acid residue is a natural occurring amino acid. In an embodiment, said additional single amino acid residue is selected from the group consisting of charged amino acids, aromatic amino acids and hydrophobic amino acids.

In an embodiment, said use in the treatment of cancer is the use in the treatment of a solid cancer.

Non-limiting examples of solid cancer include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, sarcoma, skin cancer, squamous cell carcinoma, bone cancer, melanoma, renal cell carcinoma, or kidney cancer.

In an embodiment, said solid cancer is a brain cancer, a colon cancer, a lung cancer and/or an ovarian cancer. In an embodiment, said solid cancer is a colon cancer. In another embodiment, said solid cancer is a brain cancer, preferably glioblastoma.

In embodiments, said cancer is comprised of tumor cells comprising a mutation in the TP53 tumor suppressor gene.

In an embodiment, said use in the treatment cancer comprises inhibiting proliferation of tumor cells in a subject. In an embodiment, said use in the treatment of cancer comprises inhibiting proliferation of brain cancer cells, colon cancer cells, lung cancer cells and/or ovarian cancer cells in a subject. In an embodiment, said use in the treatment of brain cancer comprises inhibiting proliferation of glioblastoma cancer cells.

In an embodiment, said use in the treatment of cancer comprises tumor growth inhibition in a subject. In an embodiment, said use in the treatment of cancer comprises tumor growth inhibition of brain cancer cells, colon cancer cells, lung cancer cells and/or ovarian cancer cells in a subject. In an embodiment, said use in the treatment of brain cancer comprises tumor growth inhibition of glioblastoma cancer cells.

In an embodiment, said use in the treatment of cancer comprises inducing and/or enhancing apoptosis in tumor cells in a subject. In an embodiment, said use in the treatment of cancer method comprises inducing and/or enhancing apoptosis in brain cancer cells, colon cancer cells, lung cancer cells and/or ovarian cancer cells in a subject. In an embodiment, said use in the treatment of brain cancer method comprises inducing and/or enhancing apoptosis in glioblastoma cancer cells.

In an embodiment, the use in the treatment of cancer comprises inhibiting angiogenesis of tumor cells in a subject. In an embodiment, use in the treatment of cancer comprises inhibiting angiogenesis of brain cancer cells, colon cancer cells, lung cancer cells and/or ovarian cancer cells in a subject. In an embodiment, use in the treatment of brain cancer comprises inhibiting angiogenesis of glioblastoma cancer cells.

In an embodiment, said use in the treatment of cancer is the use in the treatment of a solid cancer. In an embodiment, said solid cancer is a brain cancer, colon cancer, a lung cancer and/or an ovarian cancer. In an embodiment, said solid cancer is a colon cancer. In another embodiment, said solid cancer is a brain cancer. In a particular embodiment said brain cancer is glioblastoma multiforme.

In an embodiment, said cyclic peptide variant of Ang(1-7) for use in the treatment of cancer consists of the amino acid sequence:

(SEQ ID NO: 1)
Xaa1-Asp-Arg-Val-Abu/Ala-Ile-His-Abu/Ala

comprising a thioether-bridge linkage between the side chains of Abu/Ala at position 5 and Abu/Ala at position 8, and wherein Xaa1 is selected from the group consisting of Lys, Tyr, Asp, pGlu and lle.

In another embodiment, said cyclic peptide variant of Ang(1-7) for use in the treatment of cancer comprises the amino acid sequence:

(SEQ ID NO: 1)
Xaa1-Asp-Arg-Val-Abu/Ala-Ile-His-Abu/Ala

comprising a thioether-bridge linkage between the side chains of Abu/Ala at position 5 and Abu/Ala at position 8, and wherein Xaa1 is selected from the group consisting of Lys, Tyr, Asp, pGlu and ll.

In an embodiment, Xaa¹ of said cyclic peptide variant of Ang(1-7) is a D-stereoisomer.

In an embodiment, Xaa¹ of said cyclic peptide variant of Ang(1-7) is Lys.

In an embodiment, position 5 of said cyclic peptide variant of Ang(1-7) is a D-stereoisomer of Ala.

In an embodiment, position 8 of said cyclic peptide variant of Ang(1-7) is an L-stereoisomer of Ala.

In an embodiment, position 8 of said cyclic peptide variant of Ang(1-7) is an L-stereoisomer of Ala and wherein Lys is a D-stereoisomer.

In an embodiment, position 5 of said cyclic peptide variant of Ang(1-7) is a D-stereoisomer of Ala and position 8 is an L-stereoisomer of Ala.

In an embodiment, said cyclic peptide variant of Ang(1-7) has an amino acid sequence of Lys-Asp-Arg-Val-Abu/Ala-Ile-His-Abu/Ala (SEQ ID NO: 2) under the provision that the peptide does not contain two Abu (2-aminobutyric acid) residues. In another embodiment, said cyclic peptide variant of Ang(1-7) has an amino acid sequence of Lys-Asp-Arg-Val-Abu/Ala-Ile-His-Abu/Ala (SEQ ID NO: 2) under the provision that the peptide does not contain two Ala residues.

Mode of Action

In general, the cyclic peptide variant of Ang(1-7) according to the present disclosure may be used in the treatment of cancer, in which endogenous production of AT2 receptor agonists is deficient, and/or an increase in the effect of AT2 receptor agonists is desired or required, and/or a where AT2 receptors are expressed and their stimulation is desired or required, and/or when ligand-mediated upregulation of the constitutively active AT2R is desired.

Accordingly, in certain embodiments, the present disclosure provides a XcAng(1-7) for use in the treatment of cancer wherein the endogenous production of AT2 receptor agonists is deficient. In an embodiment, said AT2 receptor agonist is an Xaa1-cAngiotensin(1-7).

In an embodiment, the present disclosure provides a XcAng(1-7) for use in the treatment of cancer, wherein said XcAng(1-7) increases or enhances the effect of an AT2 receptor agonist. In an embodiment, the present disclosure provides a XcAng(1-7) for use in the treatment of cancer wherein said XcAng(1-7) stimulates or enhances AT2 receptor activity expressed on tumor and/or cancer cells.

In an embodiment, the present disclosure provides a XcAng(1-7) for use in the treatment of cancer wherein said XcAng(1-7) has selective action via AT2R and is not acting via AT1R, In another embodiment XcAng(1-7) stimulates or enhances the signaling pathways and/or mechanisms that are mediated by the AT2 receptor expressed on tumor and/or cancer cells. In an embodiment, the present disclosure provides a XcAng(1-7) for use in the treatment of cancer, wherein said cAng(1-7) specifically enhances or stimulates AT2-receptor mediated signal transduction.

In an embodiment, the present disclosure provides a XcAng(1-7) for use in the treatment of cancer, wherein said XcAng(1-7) agonizes AT2 receptor activity.

In an embodiment, the present disclosure provides a XcAng(1-7) for use in the treatment of cancer, wherein said XcAng(1-7) upregulates the expression of constitutively active AT2 receptor.

In an embodiment, the present disclosure provides a XcAng(1-7) for use in the treatment of cancer, wherein said XcAng(1-7) is an agonist of the AT2 receptor.

In an embodiment, said cancer is associated with an undesired absence or reduced AT2 receptor activity, in particular human AT2 receptor activity.

In an embodiment, said cancer is associated with an undesired absence or reduced AT2R-associated tumor suppressor (ATIP and/or SHP-1/SHP-2 and/or PLZF) expression or activity.

In an embodiment, the present disclosure provides an XcAng(1-7) for use in the treatment of cancer, wherein said cAng(1-7) stimulates or enhances AT2R-associated tumor suppressor (ATIP and/or SHP-1/SHP-2 and/or PLZF) expression or activity.

In an embodiment, the present disclosure provides an XcAng(1-7) for use in the treatment of cancer, wherein AT2R stimulation leads to upregulation of ATIP and p53, wherein the XcAng(1-7) is preferably KcAng(1-7), and the cancer is preferably, colon cancer.

In an embodiment, said AT2 receptor activity and/or said AT2R-associated tumor suppressor (ATIP and/or SHP-1/SHP-2 and/or PLZF) activity is stimulated or enhanced in vivo. In an embodiment, said activity is stimulated or enhanced in a subject after administering said XcAng(1-7).

In an embodiment, said use in the treatment of cancer is the use in the treatment of a solid cancer. In an embodiment, said solid cancer is a brain cancer, a colon cancer, a lung cancer and/or an ovarian cancer. In an embodiment, said solid cancer is a colon cancer. In another embodiment, said solid cancer is a brain cancer. In a particular embodiment said brain cancer is glioblastoma multiforme.

In an embodiment, the present disclosure provides an XcAng(1-7) for use in the treatment of cancer, wherein said XcAng(1-7) is an agonist of the AT2 receptor.

In an embodiment, the present disclosure provides an XcAng(1-7) for use in the treatment of cancer, wherein said XcAng(1-7) is an agonist of the AT2 receptor.

In an embodiment, the present disclosure provides a cyclized peptide variant of XcAng(1-7), wherein said cyclized peptide variant of XcAng(1-7), is an agonist of the AT2 receptor.

In an embodiment, the present disclosure provides a thioether-bridged peptide variant of XcAng(1-7), wherein said peptide variant is an agonist of the AT2 receptor.

In a more specific embodiment, the present disclosure provides a thioether-bridged peptide variant of XcAng(1-7) extended with an additional amino acid at the N-terminus, for use in the treatment of cancer, wherein said thioether-bridged peptide variant of Angiotensin-(1-7) extended with an additional amino acid at the N-terminus is an agonist of the AT2 receptor.

In an embodiment, said additional single amino acid residue is selected from the group consisting of charged amino acids, aromatic amino acids and hydrophobic amino acids.

In an embodiment, said additional single amino acid residue is a natural occurring amino acid. In an embodiment, said additional single amino acid residue is selected from the group consisting of charged amino acids, aromatic amino acids and hydrophobic amino acids.

In an embodiment, said peptide variant of XcAng(1-7) for use in the treatment of cancer consist of the amino acid sequence:

(SEQ ID NO: 1)
Xaa1-Asp-Arg-Val-Abu/Ala-Ile-His-Abu/Ala

comprising a thioether-bridge linkage between the side chains of Abu/Ala at position 5 and Abu/Ala at position 8, and
wherein Xaa1 is selected from the group consisting of Lys, Tyr, Asp, pGlu and Ile

In an embodiment, Xaa1 of said peptide variant of XcAng(1-7) is a D-stereoisomer.

In an embodiment, Xaa1 of said peptide variant of XcAng(1-7) is Lys.

In an embodiment, position 5 of said peptide variant of XcAng(1-7) is a D-stereoisomer of Ala.

In an embodiment, position 8 of said peptide variant of XcAng(1-7) is an L-stereoisomer of Ala.

In an embodiment, position 8 of said peptide variant of XcAng(1-7) is an L-stereoisomer of Ala and wherein position 1 is a Lys and a D-stereoisomer.

In an embodiment, position 5 of said peptide variant of XcAng(1-7) is a D-stereoisomer of Ala and position 8 is an L-stereoisomer of Ala.

In an embodiment, said peptide variant of XcAng(1-7) has an amino acid sequence of Lys-Asp-Arg-Val-Abu/Ala-Ile-His-Abu/Ala (SEQ ID NO: 2) under the provision that the peptide does not contain two Abu (2-aminobutyric acid) residues.

Methods of Treatment

The present disclosure also provides for methods of treating cancer comprising administering to a subject a therapeutically effective amount of a cyclic peptide according to the present invention.

In certain embodiments, the present disclosure provides for methods of inhibiting tumor growth comprising administering to a subject a therapeutically effective amount of a cyclic peptide according to the present disclosure.

According to a further aspect of the present disclosure, there is provided a method of treatment of cancer in which endogenous production of AT2 receptor agonists is deficient, and/or a cancer where an increase in the effect of AT2 receptor agonists is desired or required, and/or a cancer where AT2 receptors are expressed and their stimulation is desired or required, and/or wherein ligand-mediated upregulation of the constitutively active AT2R is desired.

In another embodiment, the present disclosure provides a cyclic peptide according to the present disclosure for use in the manufacture of a medicament for use in the treatment of cancer.

In an embodiment, the present disclosure provides the use of a cyclic peptide according to the present invention in the preparation of a medicament for the treatment of cancer.

In an embodiment, the present disclosure provides a cyclic peptide according to the present disclosure for use as a medicament for use in the treatment of cancer.

In an embodiment, the present disclosure provides the use of a cyclic peptide according to the present disclosure for the manufacture of a medicament for use in the treatment of cancer. In certain embodiments, the cancer is a solid cancer. In certain embodiments, the solid cancer is brain cancer, colon cancer, lung cancer and/or ovarian cancer. In certain embodiments, the subject is a human. In certain embodiments, the cancer is a resistant cancer and/or relapsed cancer.

The present disclosure provides cyclic peptides according to the present disclosure, in particular, KcAng(1-7) (SEQ ID NO:2), for use in treating brain cancer, colon cancer, lung cancer and/or ovarian cancer.

In a particular aspect, the present disclosure provides KcAng(1-7) (SEQ ID NO:2) for use in treating glioblastoma multiforme.

In another aspect, a pharmaceutical composition comprising KcAng(1-7) (SEQ ID NO:2) is provided for use in treating brain cancer, colon cancer, lung cancer and/or ovarian cancer.

In a particular aspect, a pharmaceutical composition comprising KcAng(1-7) (SEQ ID NO:2) is provided for use in treating glioblastoma multiforme.

In another embodiment, the present disclosure provides the use of a KcAng(1-7) (SEQ ID NO:2) for the preparation of a medicament for use in the treatment of brain cancer, colon cancer, lung cancer and/or ovarian cancer.

In a particular embodiment, the present disclosure provides the use of a KcAng(1-7) (SEQ ID NO:2) for the preparation of a medicament for use in the treatment of glioblastoma multiforme.

In one aspect, a method for treating brain cancer, colon cancer, lung cancer and/or ovarian cancer is provided using a cyclic peptide of the present disclosure, in particular KcAng(1-7) (SEQ ID NO:2). In a particular aspect, a method for treating glioblastoma multiforme is provided using a cyclic peptide of the present disclosure, in particular KcAng(1-7) (SEQ ID NO:2).

In certain embodiments, the resistant cancer is resistant to tyrosine kinase inhibitors, including but not limited to, EGFR inhibitors, Her2 inhibitors, Her3 inhibitors, IGFR inhibitors and Met inhibitors. In certain embodiments, said tyrosine kinase inhibitor resistant cancer is resistant to EGFR inhibitors, Her2 inhibitors, Her3 inhibitors, IGFR inhibitors and/or Met inhibitors.

For the treatment of the disease, the appropriate dosage of an cyclic peptide according to the present disclosure depends on various factors, such as the type of disease to be treated, the severity and course of the disease, the responsiveness of the disease, previous therapy, patient's clinical history, and so on. The cyclic peptide according to the present disclosure can be administered one time or over a series of treatments lasting from several days to several months, or until a cure is effected or a diminution of the disease state is achieved (e.g., reduction in tumor size).

Combination Therapy

In certain embodiments, a cyclic peptide according to the present disclosure is combined with other therapeutic agents, such as other anti-cancer agents, anti-allergic agents, anti-nausea agents (or anti-emetics), pain relievers, cytoprotective agents, and combinations thereof.

In an embodiment, a cyclic peptide according to the present disclosure is combined in a pharmaceutical combination formulation, or dosing regimen as combination therapy, with a second compound having anti-cancer properties.

The second compound of the pharmaceutical combination formulation or dosing regimen can have complementary activities to the cyclic peptide of the combination such that they do not adversely affect each other.

For example, a cyclic peptide according to the present disclosure can be administered in combination with, but not limited to, a chemotherapeutic agent, a tyrosine kinase inhibitor, a AT2-Receptor downstream signalling pathway activator, an AT1-Receptor antagonists, IAP inhibitors, Bcl2 inhibitors, Mcl1 inhibitors, and other AT2-Receptor agonists.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately (additive effect of a combination). A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. “Synergy”, “synergism” or “synergistic activity” of a combination is determined herein by the method of Clarke et al. See Clarke et al., Issues in experimental design and endpoint analysis in the study of experimental cytotoxic agents in vivo in breast cancer and other models, Breast Cancer Research and Treatment 46:255-278 (1997).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In one aspect, the present disclosure provides a cyclic peptide according to the present disclosure for the use of treating cancer by administering to a subject in need thereof a cyclic peptide according the present disclosure in combination with one or more AT1-receptor antagonists and or with one or more AT2 receptor agonists.

Angiotensin receptor antagonists which bind to the AT1 have been disclosed in inter alia European patent applications EP 409 332, EP 512 675 WO 94/27597, WO 94/02142, WO 95/23792 and WO 94/03435; and U.S. Pat. Nos. 5,091,390, 5,177,074, 5,412,097, 5,250,521, 5,260,285, 5,376,666, 5,252,574, 5,312,820, 5,330,987, 5,166,206, 5,932,575 and 5,240,928.

In an embodiment, said AT1 receptor antagonist is a sartan, e.g. Losartan and/or Candesartan.

In one aspect, the present disclosure provides a cyclic peptide according to the present disclosure for the use of treating cancer by administering to a subject in need thereof a cyclic peptide according the present disclosure in combination with one or more other AT2 receptor agonists. Peptide and non-peptide AT2 receptor agonists have been disclosed in, for example, international patent applications WO 00/38676, WO 00/56345, WO 00/09144, WO 99/58140, WO 99/52540, WO 99/46285, WO 99/45945, WO 99/42122, WO 99/40107, WO 99/40106, WO 99/39743, WO 99/26644, WO 98/33813, WO 00/02905 and WO 99/46285; U.S. Pat. No. 5,834,432; and Japanese patent application JP 143695.

Thioether-bridged peptide variants of Angiotensin-(1-7) are also known in the art. See for example Kluskens et al. (J Pharmacol Exp Ther. 2009 March; 328(3):849-54), WO 2008/130217 and WO 2012/070936.

In an aspect, the present disclosure provides a cyclic peptide according to the present disclosure for the use of treating cancer by administering to a subject in need thereof a cyclic peptide according the present disclosure in combination with one or more tyrosine kinase inhibitors, including but not limited to, EGFR inhibitors, Her2 inhibitors, Her3 inhibitors, IGFR inhibitors, and Met inhibitors.

Tyrosine kinase inhibitors include but are not limited to: Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Epidermal growth factor receptor (EGFR) inhibitors include but are not limited to, Erlotinib hydrochloride (Tarceva®), Gefitinib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PK1166, CAS 187724-61-4).

EGFR antibodies include but are not limited to, Cetuximab (Erbitux®); Panitumumab (Vectibix®); Matuzumab (EMD-72000); Trastuzumab (Herceptin®); Nimotuzumab (hR3); Zalutumumab; TheraCIM h-R3; MDX0447 (CAS 339151-96-1); and ch806 (mAb-806, CAS 946414-09-1).

Human Epidermal Growth Factor Receptor 2 (Her2 receptor) (also known as Neu, ErbB-2, CD340, or p185) inhibitors include but are not limited to, Trastuzumab (Herceptin®); Pertuzumab (Omnitarg®); trastuzumab emtansine (Kadcyla®); Neratinib (HKI-272, (2E)-N-[4-[[3-chloro-4-[(pyridin-2-yl)methoxy]phenyl]amino]-3-cyano-7-ethoxyquinolin-6-yl]-4 (dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8).

Her3 inhibitors include but are not limited to, LJM716, MM-121, AMG-888, RG7116, REGN-1400, AV-203, MP-RM-1, MM-111, and MEHD-7945A.

MET inhibitors include but are not limited to: Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)—N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU11274); (3Z)—N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1H-pyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin-3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7). IGF1R inhibitors include but are not limited to, BMS-754807, XL-228, OSI-906, GSK0904529A, A-928605, AXL1717, KW-2450, MK0646, AMG479, IMCA12, MEDI-573, and B1836845.

In another aspect, the present disclosure provides a cyclic peptide according to the present disclosure for use in the treatment of cancer by administering to a subject in need thereof a cyclic peptide according to the present disclosure in combination with one or more AT2 receptor downstream signaling pathway inhibitors, e.g. ERbB4 inhibitors.

In another aspect, the present disclosure provides a cyclic peptide according to the present disclosure for use in the treatment of cancer by administering to a subject in need thereof a cyclic peptide according to the present disclosure in combination with one or more pro-apoptotics, including but not limited to, IAP inhibitors, Bcl2 inhibitors, MCl1 inhibitors, Trail agents, Chk inhibitors.

IAP inhibitors include but are not limited to, LCL161, GDC-0917, AEG-35156, AT406, and TL32711. Other examples of IAP inhibitors include but are not limited to those disclosed in WO04/005284, WO 04/007529, WO 05/097791, WO 05/069894, WO 05/069888, WO 05/094818, US 2006/0014700, US 2006/0025347, WO 06/069063, WO 06/010118, WO 06/017295, and WO 08/134679.

BCL-2 inhibitors include but are not limited to 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab (CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinyl-pyrazolo[1,5-a]pyrimidin-7-amine (SCH900776, CAS 891494-63-6); 5-(3-Fluorophenyl)-3-ureidothiophene-2-carboxylic acid N—[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7,4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (YGRKKRRQRRRLYRSPAMPENL), and CBP501 ((d-Bpa)sws(d-Phe-F5)(d-Cha)rrrqrr).

In a further embodiment, the present invention provides a cyclic peptide according to the present disclosure for use in the treatment of cancer by administering to a subject in need thereof a cyclic peptide according to the present disclosure in combination with one or more immunomodulators (e.g., one or more of: an activator of a costimulatory molecule or an inhibitor of an immune checkpoint molecule).

In certain embodiments, the immunomodulator is an activator of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In certain embodiments, the immunomodulator is an inhibitor of an immune checkpoint molecule. In one embodiment, the immunomodulator is an inhibitor of PD-1, PD-L1, PD-L2, CTLA4, TIM3, LAG3, VISTA, BTLA, TIGIT, LAIR1, CD160, 2B4 and/or TGFR beta. In one embodiment, the inhibitor of an immune checkpoint molecule inhibits PD-1, PD-L1, LAG-3, TIM-3 or CTLA4, or any combination thereof.

Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide e.g., a soluble ligand (e.g. PD-1-Ig or CTLA-4 Ig), or an antibody or antigen-binding fragment thereof, that binds to the inhibitory molecule; e.g., an antibody or fragment thereof that binds to PD-1, PD-L1, PD-L2, CTLA4, TIM3, LAG3, VISTA, BTLA, TIGIT, LAIR1, CD160, 2B4 and/or TGFR beta, or a combination thereof.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immunomodulators, e.g., in combination with an inhibitor of LAG-3, TIM-3 or CTLA4. In an exemplary embodiment, the inhibitor of PD-1 or PD-L1, e.g., the anti-PD-1 or PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule. Other combinations of immunomodulators with a PD-1 inhibitor (e.g., one or more of PD-L2, CTLA4, TIM3, LAG3, VISTA, BTLA, TIGIT, LAIR1, CD160, 2B4 and/or TGFR) are also within the present disclosure.

In an embodiment, the PD-1 inhibitor is an anti-PD-1 antibody chosen from Nivolumab, Pembrolizumab or Pidilizumab. Nivolumab and other human monoclonal antibodies that specifically bind to PD1 are disclosed in U.S. Pat. No. 8,008,449 and WO2006/121168.

In other embodiments, the anti-PD-1 antibody is Pembrolizumab (see Hamid, O. et al. (2013) New England Journal of Medicine 369 (2): 134-44, WO 2009/114335; U.S. Pat. No. 8,354,509). Other anti-PD1 antibodies are disclosed in U.S. Pat. No. 8,609,089, US Pub. No. 2010028330, and/or US Pub. No. 20120114649.

In certain embodiments, the PD-1 inhibitor is an immunoadhesin (e.g., an immunoadhesin comprising an extracellular or PD-1 binding portion of PD-L1 or PD-L2 fused to a constant region e.g., an Fc region of an immunoglobulin sequence. In some embodiments, the PD-1 inhibitor is AMP-224.

In certain embodiments, the PD-L1 inhibitor is anti-PD-L1 antibody. In some embodiments, the anti-PD-L1 inhibitor is chosen from YW243.55.S70, MPDL3280A, MEDI-4736, or MDX-1105MSB-0010718C (also referred to as A09-246-2) disclosed in WO 2013/0179174.

In an embodiment, the PD-L1 inhibitor is MDX-1105 described in WO 2007/005874. In an embodiment, the PD-L1 inhibitor is YW243.55.S70 (WO 2010/077634) In an embodiment, the PD-L1 inhibitor is MDPL3280A (Genentech/Roche). MDPL3280A and other human monoclonal antibodies to PD-L1 are disclosed in U.S. Pat. No. 7,943,743 and U.S Publication No.: 20120039906.

In other embodiments, the PD-L2 inhibitor is AMP-224 (Amplimmune; WO 2010/027827 and WO2011/066342).

In a particular aspect, a cyclic peptide according to the present disclosure for the use of treating a brain cancer, such as glioblastoma multiforme or anaplastic astrocytoma is combined with temozolomide (TMZ), sold under the brand name Temodar or Temodal®.

In a particular aspect, the present disclosure provides KcAng(1-7) for the use in treating glioblastoma multiforme, whereby KcAng(1-7) is to be administered in combination with temozolomide (TMZ). The disclosure also provides temozolomide for use in treating glioblastoma multiforme, whereby temozolomide is to be administered in combination with KcAng(1-7).

Further, a method is provided for treating a brain cancer, such as glioblastoma multiforme or anaplastic astrocytoma using KcAng(1-7) in combination with temozolomide (TMZ) and/or an AT1-Antagonist, preferably Losartan.

In another aspect, the present disclosure provides KcAng(1-7) for the use in treating glioblastoma multiforme, whereby KcAng(1-7) is to be administered in combination with an AT1-Antagonist, preferably Losartan. The disclosure also provides an AT1-Antagonist, preferably Losartan, for use in treating glioblastoma multiforme, whereby Losartan is to be administered in combination with KcAng(1-7).

In a particular aspect a synergistic combination of KcAng(1-7) and temozolomide (TMZ) for the use in treating glioblastoma multiforme is provided.

In another aspect a synergistic combination of KcAng(1-7) and temozolomide (TMZ) for the use in treating glioblastoma multiforme is provided, wherein the synergistic effect is on tumor weight reduction.

### WORKING EXAMPLES

### Example 1

In vivo evaluation of the effect of KcAng(1-7) on the growth of different human patient-derived xenografts (PDX) cancer models.

### 1.1 In Vivo Study Design

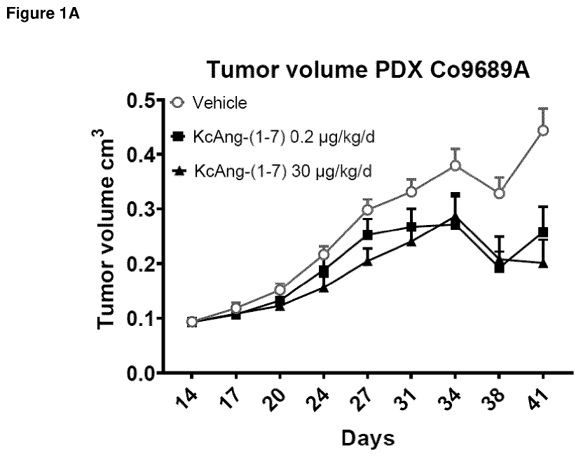

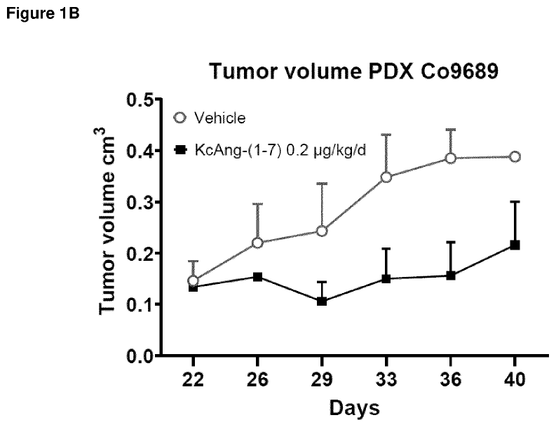

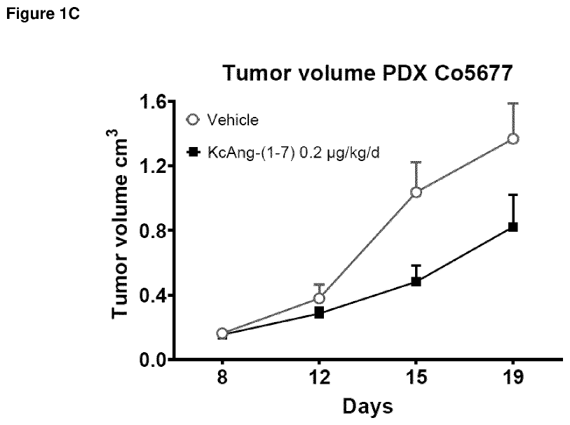

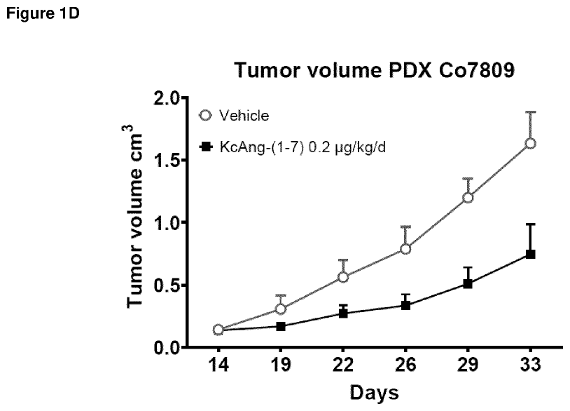

In vivo studies were conducted at EPO GmbH Berlin. Large studies (FIG. 1A, 2, 3, 4, 5) comprised 3 groups of 10 mice/group and 5 animals as reserve, as outlined in Table 1. At the beginning of the study, 30 mice were inoculated with respective PDX tumors (PDX tumors are summarized in Table 2). Smaller studies (FIG. 1B, 1C, 1D) comprised 2 groups of mice of 3 mice per group. The animals were allocated to the various treatment groups (e.g. 0.2 μg/kg and 30 μg/kg) with a maximum of 10 animals per group. Subsequently, animals were observed for tumor growth two times a week starting at day 0 of sufficient tumor size reached after tumor inoculation. The groups received subcutaneous (s.c.) drug injection (for max. 28 days) as outlined in the Table 1.

NMRI nu/nu mice were used in this study. The animals were female at age of 6 to 8 weeks. The respective PDX tumors were inoculated subcutaneously (s.c.), and the tumors were grown to achieve palpable tumor sizes before start of the respective treatments. The PDX included breast (MaCa4151), colon (Co9689A), head & neck (HN10309), lung (Lu7433) and ovarian (OvCa13329) cancer (Table 2).

TABLE 2
Experimental design of the in vivo study to test KcAng-(1-7) in PDX tumor models

| | No. | | Application | Sequence | | Dose |
|---|---|---|---|---|---|---|
| Group | mice | Treatment | Route | Days | Hours | mg/kg/inj. |
| A | 10 | Vehicle | s.c. | qd × 28 | 09:00 | |
| B | 10 | KcAng-(1-7) | s.c. | qd × 28 | 09:00 | 0.2 kg/kg |
| C | 10 | KcAng-(1-7) | s.c. | qd × 28 | 09:00 | 30 kg/kg |
| D | 5 | Reserve | | | | |

TABLE 3
Overview PDX models of human solid cancers of
different origin used for testing of KcAng-(1-7)

| PDX model | Tumor type |
|---|---|
| Co9689A | Colon adenocarcinoma |
| HN10309 | Head&Neck carcinoma; squamous cell carcinoma, HPV pos. |
| Lu7433 | Lung carcinoma; squamous cell carcinoma |
| MaCa4151 | Invasive ductal carcinoma of the breast, ER/PR neg. |
| OvCa13329 | Tubular adenocarcinoma of the ovary |

Treatment with KcAng(1-7) was started with palpable tumors after the randomization of animals regarding tumor volume. KcAng(1-7) was applied s.c. by daily injections at 0.2 g/kg or 30 μg/kg dose for a maximum of 28 days (or earlier as defined by the humane end point for the study).

KcAng(1-7) was prepared as follows: The compounds was suspended in sterile PBS resulting in a clear solution. The remains of this formulation were stored at 4° C.

Tumor growth stimulatory or inhibitory activity of the KcAng(1-7) was evaluated by determination of tumor volumes (TV). During the study, tumor volumes and body weight were measured twice a week, and the ratio of the mean tumor volumes between the compound-treated group and the vehicle-treated group (T/C) was calculated.

The tumor volume and T/C were calculated according to the following equations:

Tumor volume=(tumor width)×(tumor width)×(tumor length)+2

T/C=T÷C
T: Mean estimated tumor volume of the compound-treated group
C: Mean estimated tumor volume of the vehicle-treated group

For further analyses, tumors were collected after sacrifice of anesthetized mice. The tumor samples were snap frozen or formalin fixed (FFPE).

### 1.2 Results of the In Vivo Studies

In the following, data for KcAng(1-7) are depicted for each tumor entity (represented by one PDX) including entity specific conclusions drawn from the experimental data obtained.

### 1.3 Colon Carcinoma PDX Co9689A

For the colon carcinoma PDX model Co9689A, treatment with KcAng(1-7) was performed for 28 days. Overall, treatment was well tolerated as determined by mean body weight assessment (data not shown).

Treatment with KcAng(1-7) resulted in a statistically significant and dose dependent reduction in tumor growth with a reduction in tumor volume of 58% for the dose of 0.2 μg/kg and of 45% for the dose of 30 μg/kg at day 28 (day 41 of study). Reduction in tumor growth was of highest significance for a dose of 30 μg/kg.

Mean values for tumor volume measurements, TIC and RTV values are summarized in Table 3.

TABLE 4
Mean values of tumor volumes, T/C and RTV of the Co9689A PDX model during
treatment with KcAng(1-7) (treatment for 28 days).

| | Meas. | | 1 | 2 | 3 | 4 | 5 | 6 | 7 | 8 | 9 |
|---|---|---|---|---|---|---|---|---|---|---|---|
| A | (n) | | 10 | 10 | 10 | 10 | 10 | 10 | 10 | 10 | 10 |
| | Meas. | | Gr. A | Gr. A | Gr. A | Gr. A | Gr. A | Gr. A | Gr. A | Gr. A | Gr. A |
| | | | M1 | M2 | M3 | M4 | M5 | M6 | M7 | M8 | M9 |
| | Tumor | Median | 0.084 | 0.122 | 0.148 | 0.216 | 0.311 | 0.326 | 0.37 | 0.337 | 0.442 |
| | Vol. | Mean | 0.093 | 0.118 | 0.151 | 0.216 | 0.298 | 0.331 | 0.379 | 0.328 | 0.443 |
| | [cm³] | [S.D.] | 0.0258 | 0.0304 | 0.0382 | 0.047 | 0.0605 | 0.0706 | 0.0975 | 0.0931 | 0.1266 |
| | RTV | Median | 1 | 1.2 | 1.7 | 2.3 | 3.3 | 3.9 | 4.2 | 3.4 | 4.8 |
| | | Mean | 1 | 1.3 | 1.7 | 2.4 | 3.3 | 3.7 | 4.1 | 3.7 | 4.8 |
| | Meas. | | Gr. B | Gr. B | Gr. B | Gr. B | Gr. B | Gr. B | Gr. B | Gr. B | Gr. B |
| | | | M1 | M2 | M3 | M4 | M5 | M6 | M7 | M8 | M9 |
| B | (n) | | 10 | 10 | 10 | 10 | 10 | 10 | 10 | 10 | 10 |
| | Tumor | Median | 0.09 | 0.108 | 0.126 | 0.139 | 0.234 | 0.238 | 0.214 | 0.178 | 0.219 |
| | Vol. | Mean | 0.093 | 0.107 | 0.132 | 0.188 | 0.252 | 0.267 | 0.272 | 0.192 | 0.257 |
| | [cm³] | [S.D.] | 0.0198 | 0.0202 | 0.045 | 0.0963 | 0.0929 | 0.1053 | 0.1614 | 0.0926 | 0.1486 |
| | RTV | Median | 1 | 1.2 | 1.4 | 1.9 | 2.6 | 2.7 | 2.8 | 1.9 | 2.5 |
| | | Mean | 1 | 1.2 | 1.4 | 2 | 2.7 | 2.8 | 3.2 | 2 | 2.7 |
| | T/C [%] | | 99.5 | 90.3 | 87.4 | 87.1 | 84.6 | 80.7 | 71.6 | 58.6 | 58.1 |
| | Meas. | | Gr. C | Gr. C | Gr. C | Gr. C | Gr. C | Gr. C | Gr. C | Gr. C | Gr. C |
| | | | M1 | M2 | M3 | M4 | M5 | M6 | M7 | M8 | M9 |
| C | (n) | | 10 | 10 | 10 | 10 | 10 | 10 | 10 | 10 | 10 |
| | Tumor | Median | 0.097 | 0.105 | 0.111 | 0.125 | 0.184 | 0.199 | 0.213 | 0.155 | 0.134 |
| | Vol. | Mean | 0.093 | 0.108 | 0.122 | 0.156 | 0.205 | 0.24 | 0.287 | 0.208 | 0.201 |
| | [cm³] | [S.D.] | 0.0178 | 0.0208 | 0.0406 | 0.0647 | 0.0717 | 0.0882 | 0.1302 | 0.1317 | 0.1338 |
| | RTV | Median | 1 | 1.2 | 1.3 | 1.5 | 2.3 | 2.4 | 2.8 | 1.9 | 1.8 |
| | | Mean | 1 | 1.2 | 1.3 | 1.7 | 2.2 | 2.5 | 3 | 2.2 | 2.1 |
| | T/C [%] | | 99.5 | 91.5 | 80.9 | 72.1 | 68.6 | 72.5 | 75.6 | 63.3 | 45.3 |

1.4 Head & Neck Carcinoma PDX Model HN10309

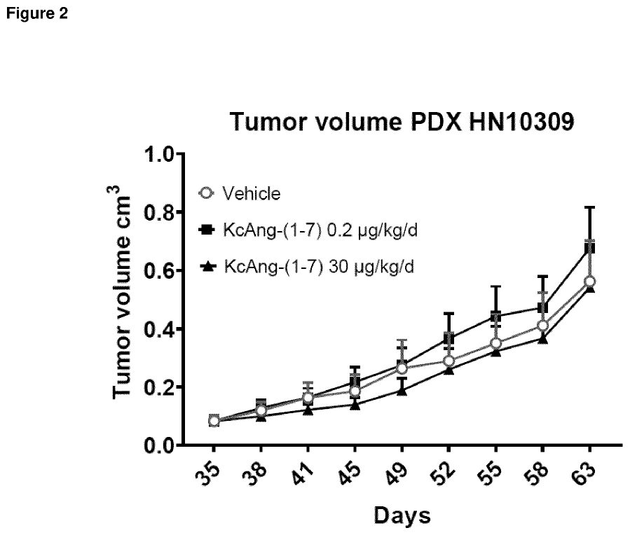

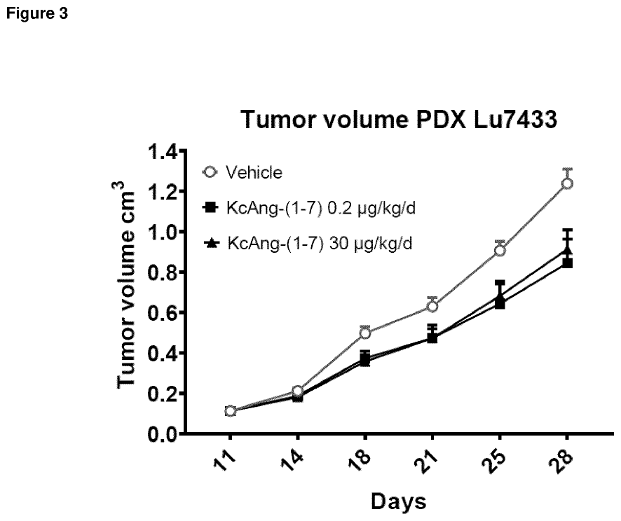

For the head & neck carcinoma PDX model, treatment with KcAng(1-7) was performed for 28 days and was well tolerated as determined by mean body weights (data not shown). Treatment with the KcAng(1-7) did not result in a reduction or increase in tumor volume as reflected by the optT/C values of 100 to 126% for the dose of 0.2 μg/kg and of 71% for the dose of 30 μg/kg at day 14 of the treatment. Table 4 summarizes the mean values of tumor volume measurement TIC and RTV values. FIG. 2 depicts tumor volume growth over time.

TABLE 5
Mean values of tumor volumes, T/C and RTV of the HN10309 PDX model during
treatment with KcAng(1-7) (treatment for 28 days).

| | Day: | | 35 | 38 | 41 | 45 | 49 | 52 | 55 | 58 | 63 |
|---|---|---|---|---|---|---|---|---|---|---|---|
| | Meas. | | 1 | 2 | 3 | 4 | 5 | 6 | 7 | 8 | 9 |
| A | (n) | | 10 | 10 | 10 | 10 | 10 | 10 | 10 | 10 | 10 |
| | Tumor | Median | 0.071 | 0.093 | 0.095 | 0.096 | 0.135 | 0.147 | 0.235 | 0.276 | 0.345 |
| | Vol. | Mean | 0.083 | 0.119 | 0.163 | 0.186 | 0.264 | 0.289 | 0.35 | 0.41 | 0.562 |
| | [cm³] | [S.D.] | 0.0574 | 0.0947 | 0.1597 | 0.1825 | 0.308 | 0.3058 | 0.3172 | 0.3565 | 0.4401 |
| | RTV | Median | 1 | 1.4 | 1.9 | 2.1 | 2.4 | 3.1 | 3.7 | 4.8 | 7.3 |
| | | Mean | 1 | 1.4 | 1.8 | 2.1 | 2.8 | 3.2 | 4.3 | 5.1 | 6.9 |
| | Meas. | | Gr. B | Gr. B | Gr. B | Gr. B | Gr. B | Gr. B | Gr. B | Gr. B | Gr. B |
| | | | M1 | M2 | M3 | M4 | M5 | M6 | M7 | M8 | M9 |
| B | (n) | | 10 | 10 | 10 | 10 | 10 | 10 | 10 | 10 | 10 |
| | Tumor | Median | 0.064 | 0.085 | 0.14 | 0.158 | 0.209 | 0.303 | 0.366 | 0.437 | 0.64 |
| | Vol. | Mean | 0.083 | 0.128 | 0.164 | 0.217 | 0.275 | 0.367 | 0.443 | 0.473 | 0.677 |
| | [cm³] | [S.D.] | 0.0543 | 0.0868 | 0.1001 | 0.1599 | 0.1883 | 0.2668 | 0.3227 | 0.3391 | 0.4389 |
| | RTV | Median | 1 | 1.7 | 2.1 | 2.5 | 3.2 | 4 | 4.8 | 4.9 | 7.3 |
| | | Mean | 1 | 1.6 | 2.1 | 2.6 | 3.3 | 4.4 | 5.4 | 5.8 | 8.9 |
| | T/C [%] | | 100.1 | 107.9 | 100.6 | 116.7 | 104.4 | 126.7 | 126.5 | 115.2 | 120.4 |
| | Meas. | | Gr. C | Gr. C | Gr. C | Gr. C | Gr. C | Gr. C | Gr. C | Gr. C | Gr. C |
| | | | M1 | M2 | M3 | M4 | M5 | M6 | M7 | M8 | M9 |
| C | (n) | | 10 | 10 | 10 | 10 | 10 | 10 | 10 | 10 | 10 |
| | Tumor | Median | 0.077 | 0.092 | 0.117 | 0.135 | 0.187 | 0.236 | 0.283 | 0.303 | 0.505 |
| | Vol. | Mean | 0.083 | 0.099 | 0.122 | 0.139 | 0.188 | 0.26 | 0.323 | 0.367 | 0.541 |
| | [cm³] | [S.D.] | 0.0468 | 0.0422 | 0.0593 | 0.0756 | 0.1321 | 0.2273 | 0.2715 | 0.3148 | 0.4685 |
| | RTV | Median | 1 | 1.3 | 1.5 | 1.7 | 2.2 | 2.9 | 3.5 | 4 | 5.8 |
| | | Mean | 1 | 1.3 | 1.6 | 1.8 | 2.2 | 2.8 | 3.5 | 3.9 | 5.6 |
| | T/C [%] | | 99.5 | 83.5 | 74.6 | 75 | 71.3 | 89.8 | 92.1 | 89.3 | 96.3 |

1.5 Lung Carcinoma PDX Lu7433

For the lung carcinoma PDX model, treatment with KcAng(1-7) was performed for 19 days, which was well tolerated as determined by mean body weight (data not shown). The study was terminated before 28 days due to the reach of the humane end point (tumor volume>1.5 cm³). Overall, treatment with KcAng(1-7) resulted in a moderate but significant decrease in tumor growth, reflected by the optT/C values of 68.2% for the dose of 0.2 μg/kg and of 71.9% for the dose of 30 μg/kg. Table 5 summarizes the mean values of tumor volume measurement TIC and RTV values.

TABLE 6
Mean values of tumor volumes, T/C and RTV of the Lu7433 PDX model during
treatment with KcAng(1-7) (treatment for 19 days).

| Group | Day: | | 11 | 14 | 18 | 21 | 25 | 28 |
|---|---|---|---|---|---|---|---|---|
| A | Meas. | | 1 | 2 | 3 | 4 | 5 | 6 |
| | (n) | | 10 | 10 | 10 | 10 | 10 | 10 |
| | Tumor | Median | 0.108 | 0.224 | 0.511 | 0.641 | 0.93 | 1.187 |
| | Vol. | Mean | 0.112 | 0.211 | 0.496 | 0.628 | 0.906 | 1.237 |
| | [cm³] | [S.D.] | 0.0211 | 0.0452 | 0.1099 | 0.1414 | 0.1411 | 0.2285 |
| | RTV | Median | 1 | 1.9 | 4.3 | 5.7 | 8.1 | 11.8 |
| | | Mean | 1 | 1.9 | 4.4 | 5.6 | 8.2 | 11.3 |
| | Meas. | | Gr. B M1 | Gr. B M2 | Gr. B M3 | Gr. B M4 | Gr. B M5 | Gr. B M6 |
| B | (n) | | 10 | 10 | 10 | 10 | 10 | 10 |
| | Tumor | Median | 0.109 | 0.19 | 0.412 | 0.454 | 0.625 | 0.808 |
| | Vol. | Mean | 0.112 | 0.187 | 0.373 | 0.473 | 0.643 | 0.844 |
| | [cm³] | [S.D.] | 0.0221 | 0.0425 | 0.1081 | 0.1987 | 0.3066 | 0.3687 |
| | RTV | Median | 1 | 1.8 | 3.4 | 4.3 | 5.6 | 8.1 |
| | | Mean | 1 | 1.7 | 3.3 | 4.2 | 5.7 | 7.6 |
| | T/C [%] | | 100.1 | 88.4 | 75.1 | 75.3 | 71 | 68.2 |
| | Meas. | | Gr. C M1 | Gr. C M2 | Gr. C M3 | Gr. C M4 | Gr. C M5 | Gr. C M6 |
| C | (n) | | 10 | 10 | 10 | 10 | 10 | 10 |
| | Tumor | Median | 0.099 | 0.168 | 0.376 | 0.509 | 0.719 | 0.991 |
| | Vol. | Mean | 0.112 | 0.18 | 0.357 | 0.472 | 0.682 | 0.912 |
| | [cm³] | [S.D.] | 0.0224 | 0.0607 | 0.0945 | 0.1503 | 0.2314 | 0.3027 |
| | RTV | Median | 1 | 1.6 | 3.1 | 4.1 | 5.9 | 8.2 |
| | | Mean | 1 | 1.6 | 3.2 | 4.3 | 6.1 | 8.2 |
| | T/C [%] | | 99.7 | 85.1 | 71.9 | 75.1 | 75.3 | 73.7 |

### 1.6 Breast Carcinoma PDX MaCa4253

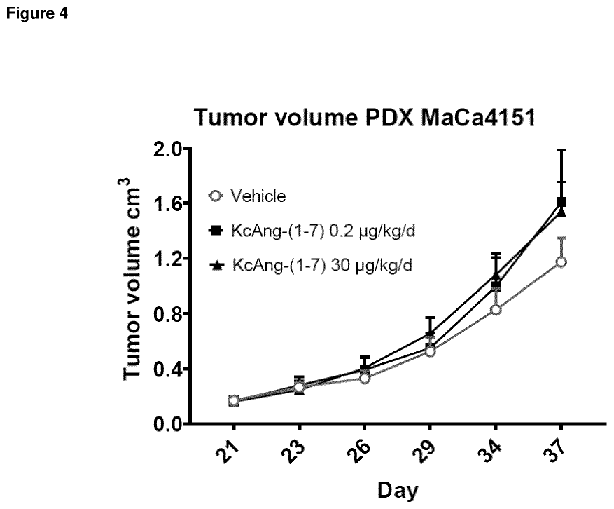

For the breast carcinoma PDX model, treatment with KcAng(1-7) was performed for 17 days and was also well tolerated as determined by mean body weight (data not shown). The study was terminated before 28 days due to the reach of the humane end point regarding tumor volume in the vehicle group (tumor volume>1.5 cm³). Treatment with KcAng(1-7) did not resulted in an increase of tumor growth or reduced tumor growth. This is also reflected by the statistical analyses of the data shown in FIG. 4 and Table 6.

TABLE 7
Statistical analysis of impact of KcAng(1-7) treatment on tumor growth in the
MaCa4151 PDX model

| | Meas. | | 1 | 2 | 3 | 4 | 5 | 6 |
|---|---|---|---|---|---|---|---|---|
| Group | Day: | | 21 | 23 | 26 | 29 | 34 | 37 |
| A | (n) | | 7 | 7 | 7 | 7 | 7 | 7 |
| | Tumor | Median | 0.149 | 0.216 | 0.269 | 0.496 | 0.698 | 1.112 |
| | Vol. | Mean | 0.171 | 0.267 | 0.331 | 0.524 | 0.828 | 1.173 |
| | [cm³] | [S.D.] | 0.0636 | 0.1065 | 0.1509 | 0.2812 | 0.408 | 0.4577 |
| | RTV | Median | 1 | 1.6 | 1.9 | 3.5 | 4.9 | 7.8 |
| | | Mean | 1 | 1.6 | 2 | 3.1 | 4.9 | 7.1 |
| | Meas. | | Gr. B M1 | Gr. B M2 | Gr. B M3 | Gr. B M4 | Gr. B M5 | Gr. B M6 |
| B | (n) | | 6 | 6 | 6 | 6 | 6 | 6 |
| | Tumor | Median | 0.131 | 0.241 | 0.368 | 0.491 | 1.021 | 1.452 |
| | Vol. | Mean | 0.163 | 0.281 | 0.392 | 0.55 | 0.995 | 1.607 |
| | [cm³] | [S.D.] | 0.0872 | 0.1482 | 0.2155 | 0.2699 | 0.5149 | 0.9219 |
| | RTV | Median | 1 | 1.6 | 2.2 | 3.2 | 5.2 | 7.6 |
| | | Mean | 1 | 1.7 | 2.4 | 3.4 | 6.2 | 10.5 |
| | T/C [%] | | 95.7 | 105.2 | 118.4 | 104.9 | 120.2 | 137 |
| | Meas. | | Gr. C M1 | Gr. C M2 | Gr. C M3 | Gr. C M4 | Gr. C M5 | Gr. C M6 |
| C | (n) | | 7 | 7 | 7 | 7 | 7 | 7 |
| | Tumor | Median | 0.157 | 0.165 | 0.383 | 0.671 | 1.072 | 1.522 |
| | Vol. | Mean | 0.162 | 0.248 | 0.405 | 0.655 | 1.081 | 1.539 |
| | [cm³] | [S.D.] | 0.0673 | 0.1661 | 0.2191 | 0.3047 | 0.4168 | 0.57 |
| | RTV | Median | 1 | 1.5 | 2.5 | 4 | 6.5 | 9.1 |
| | | Mean | 1 | 1.5 | 2.4 | 4 | 6.8 | 9.8 |
| | T/C [%] | | 94.8 | 92.6 | 122.3 | 125 | 130.6 | 131.2 |

### 1.7 Ovarian Carcinoma PDX OvCa23329

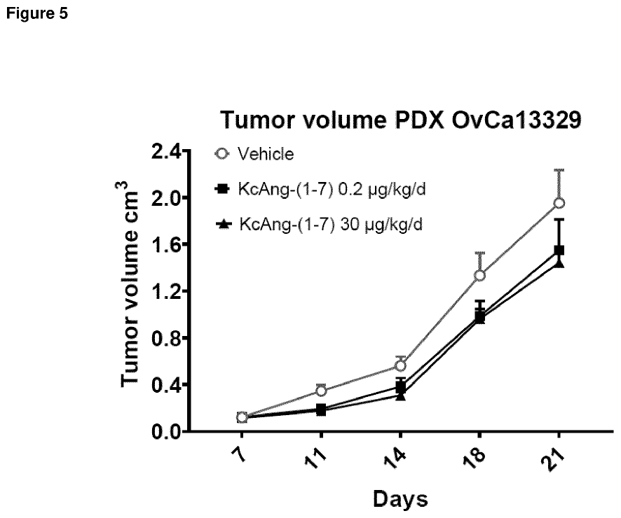

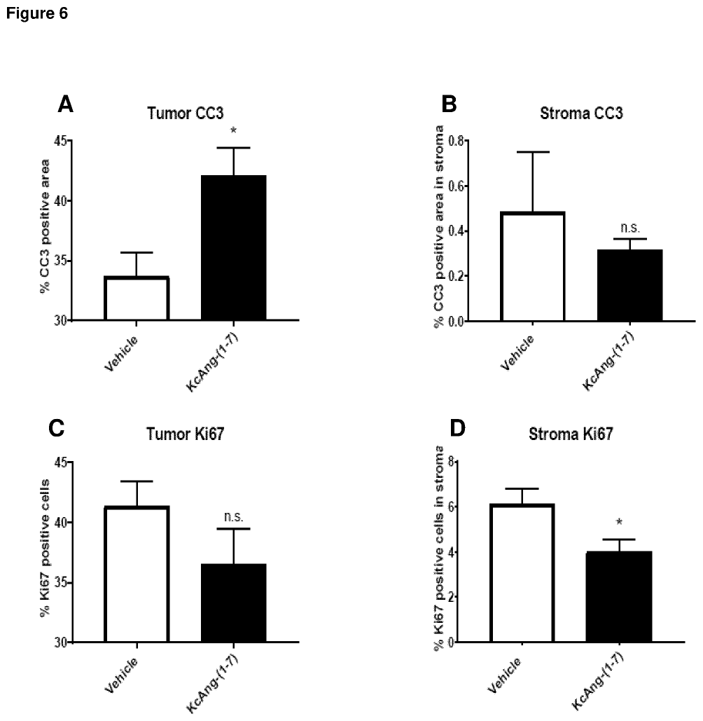

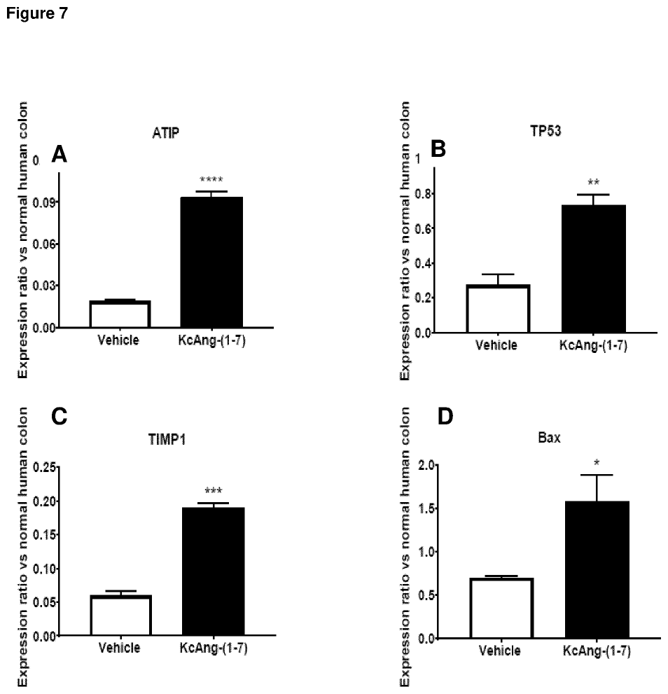

For the ovarian carcinoma PDX model the treatment with KcAng(1-7) was performed for 15 days and was also well tolerated as determined by mean body weight (data not shown). This study had to be terminated before 28 days of proposed treatment due to the reach of the human end point (tumor volume>1.5 cm³). Treatment with KcAng(1-7) did not promote tumor growth. The treatment resulted in a significant reduction in tumor volume, reflected by the optT/C values of 55.8% for the dose of 0.2 μg/kg and of 51% for the dose of 30 μg/kg at day 11 of the treatment. This is also reflected by the statistical analyses of the data shown in FIG. 5 and Table 7.

TABLE 8
Statistical analysis of impact of KcAng(1-7) treatment on tumor growth in the
OvCa13329 PDX model

| | Meas. | | 1 | 2 | 3 | 4 | 5 |
|---|---|---|---|---|---|---|---|
| Group | Day: | | 7 | 11 | 14 | 18 | 21 |
| A | (n) | | 10 | 10 | 10 | 10 | 10 |
| | Tumor | Median | 0.109 | 0.31 | 0.467 | 1.158 | 1.58 |
| | Vol. | Mean | 0.12 | 0.345 | 0.561 | 1.333 | 1.952 |
| | [cm³] | [S.D.] | 0.0249 | 0.1707 | 0.2527 | 0.6026 | 0.8934 |
| | RTV | Median | 1 | 2.4 | 4.1 | 9.5 | 14.4 |
| | | Mean | 1 | 2.9 | 4.9 | 11.6 | 17.1 |
| | Meas. | | Gr. B M1 | Gr. B M2 | Gr. B M3 | Gr. B M4 | Gr. B M5 |
| B | (n) | | 10 | 10 | 10 | 10 | 10 |
| | Tumor | Median | 0.123 | 0.181 | 0.337 | 1.015 | 1.398 |
| | Vol. | Mean | 0.122 | 0.192 | 0.384 | 0.985 | 1.547 |
| | [cm³] | [S.D.] | 0.0312 | 0.0882 | 0.2281 | 0.4071 | 0.8367 |
| | RTV | Median | 1 | 1.5 | 3 | 8 | 11.9 |
| | | Mean | 1 | 1.6 | 3.1 | 8.5 | 13.4 |
| | T/C [%] | | 102.4 | 55.8 | 68.5 | 73.9 | 79.3 |
| | Meas. | | Gr. C M1 | Gr. C M2 | Gr. C M3 | Gr. C M4 | Gr. C M5 |
| C | (n) | | 10 | 10 | 10 | 10 | 10 |
| | Tumor | Median | 0.115 | 0.182 | 0.33 | 0.995 | 1.503 |
| | Vol. | Mean | 0.116 | 0.176 | 0.308 | 0.962 | 1.442 |
| | [cm³] | [S.D.] | 0.0278 | 0.0499 | 0.0933 | 0.2754 | 0.4168 |
| | RTV | Median | 1 | 1.6 | 2.7 | 8.4 | 13 |
| | | Mean | 1 | 1.6 | 2.6 | 8.4 | 12.6 |
| | T/C [%] | | 97 | 51.1 | 54.9 | 72.2 | 73.9 |

### 1.8 Conclusions

The PDX in vivo studies were performed to evaluate the efficacy of compound KcAng(1-7) on tumor growth in five different human PDX cancer models of solid cancer (breast cancer MaCa4151, colon cancer Co9689A, head & neck cancer HN10309, lung cancer Lu7433, ovarian cancer OvCa13329).

The study revealed good tolerability of the drug at both doses used (0.2 and 30 mg/kg) in all models tested, which were all performed in female NMRI nu/nu mice.

The study revealed no tumor growth stimulatory effects in the tested PDX models.

More importantly, in lung, colon and ovarian cancer PDX models, a significant inhibition of tumor growth was observed, even at the lowest dose of 0.2 μg/kg/d tested.

This finding clearly shows an anti-tumoral activity of KcAng(1-7) in human cancer models.

### Example 2

### 2.1 Mode of Action and Involved Signaling Pathways

To investigate the mode of action and the involved signaling pathways, in the colon carcinoma PDX model that showed a remarkable retarded tumor growth (˜40%) upon treatment as described in Example 1, global kinase activity profiling of the KcAng(1-7)-treated and vehicle-treated PDX samples was performed. In particular, to determine the effect of treatment on kinase activities, Tyrosine (PTK) and Serine/Threonine (STK) PamChip®-based kinase activity profiling in lysates from colon carcinoma PDX was performed by PamGene International B.V., The Netherlands.

PamGene® kinase activity profiles were measured for vehicle-treated colon cancer PDX tumors (control, n=10) and KcAng(1-7)-treated colon cancer PDX tumors (treatment, n=10).

Frozen tissues of tumor samples were processed to make lysates followed by determination of tyrosine kinase and serine/threonine kinase activity profiles. Datasets were analyzed by PamGene bioinformatics toolbox, for example by using PamGene's method called Upstream Kinase Analysis.

### 2.2 Results

Kinase Score was used for ranking kinases based on their significance and specificity in terms of the set of peptides used for the corresponding kinase. Kinase statistics indicates the overall change of the peptide set that represents the kinase. Kinase statistics value<0 indicates higher activity in vehicle-treated tumors.

TABLE 8
Kinase Score (PTK): vehicle-treated vs KcAng(1-7)-treated

| Rank | Kinase name | Kinase score | Kinase statistic |
|---|---|---|---|
| 1 | LMR1 | 4.10 | −0.71 |
| 2 | HER2 | 2.68 | −0.47 |
| 3 | EPHA4 | 2.55 | −0.52 |
| 4 | FER | 2.49 | −0.51 |
| 5 | FGFR1 | 2.39 | −0.49 |
| 6 | EPHA3 | 2.38 | −0.55 |
| 7 | CCK4/PTK7 | 2.23 | −0.58 |
| 8 | MET | 2.16 | −0.46 |
| 9 | HER4 | 2.11 | −0.47 |
| 10 | TXK | 2.05 | −0.46 |
| 11 | TYRO3/SKY | 2.03 | −0.45 |
| 12 | EPHA1 | 2.01 | −0.52 |
| 13 | EPHA2 | 1.96 | −0.50 |
| 14 | FGFR3 | 1.89 | −0.48 |
| 15 | FES | 1.84 | −0.47 |
| 16 | FGFR2 | 1.82 | −0.47 |
| 17 | TEC | 1.79 | −0.44 |
| 18 | MER | 1.74 | −0.44 |
| 19 | EPHA8 | 1.71 | −0.51 |
| 20 | JAK1~B | 1.70 | −0.49 |

TABLE 9
Kinase Score (STK): vehicle-treated vs KcAng(1-7)-treated

| Rank | Kinase name | Kinase score | Kinase statistic |
|---|---|---|---|
| 1 | CDK2 | 5.40 | −0.88 |
| 2 | ERK5 | 5.40 | −0.90 |
| 3 | CDKL2 | 4.62 | −0.98 |
| 4 | ERK1 | 4.44 | −0.82 |
| 5 | CDKL5 | 4.21 | −0.82 |
| 6 | CDK9 | 4.19 | −0.83 |
| 7 | p38[beta] | 4.14 | −0.84 |
| 8 | JNK3 | 4.08 | −0.77 |
| 9 | JNK1 | 3.92 | −0.77 |
| 10 | ERK2 | 3.84 | −0.79 |
| 11 | CDC2/CDK1 | 3.79 | −0.80 |
| 12 | JNK2 | 3.67 | −0.77 |
| 13 | MAPK14 | 3.54 | −0.79 |
| 14 | p38[gamma] | 3.47 | −0.82 |
| 15 | PCTAIRE2 | 3.44 | −0.74 |
| 16 | RSK3 | 3.42 | −0.75 |
| 17 | CDK4 | 3.11 | −0.80 |
| 18 | CDK5 | 3.11 | −0.80 |
| 19 | CDK6 | 3.10 | −0.80 |
| 20 | RAF1 | 2.97 | −0.74 |

In addition, three different pathway analyses (GeneGo, Enrichr and Proteome Maps) on the combined PTK and STK dataset with Uniprot IDs of proteins and PamChip log fold changes were performed. Enrichr analysis with input data of n=62 proteins (with a cutoff of p<0.10) that are downregulated in KcAng(1-7) treated tumors revealed the following pathways involved (only the top 10 results of KEGG pathways are listed): MAPK, PI3K-Akt, Rap1, Ras, neurotropin, prostate cancer, HIF-1, proteoglycans in cancer, cGMP-PKG, VEGF signaling pathway.

### 2.3. Conclusions

Based on the PamChip® data, key kinases and their related pathways were identified. In particular, a specific kinome inhibition, comprising the inhibited kinases LMR1 (AATYK), HERs, FGFRs (PTK) and CDKs, ERKs, MAPKs, JNKs (STK) were identified in KcAng(1-7)-treated colon PDX tumor lysates. It was found that the major common pathways that are altered in KcAng(1-7)-treated colon tumor are the MAPK signaling pathway (MAPK1, MAPK3, MAPK7, MAPK12 and AKT1), and the Ras signaling pathway (FGFRs, FLT1/VEGFR1, CSF1R).

### Example 3

In vivo evaluation of antitumor efficacy of KcAng(1-7) in combination with temozolomide or losartan in Glioblastoma multiforme (GBM) cell culture-derived xenograft models (CDX)

### 3.1 In Vivo Study Design

Effects of KcAng(1-7) were evaluated in vivo in a subcutaneous GBM xenograft model in nude mice. Insurgence of neurologic phenomena and differences in the survival rates were evaluated. Human GBM cell lines U87MG and U251MG were used. Cell lines were grown in DMEM nutrient mixture supplemented with 10% fetal bovine serum, 1% penicillin/streptomycin, and 25 mM HEPES buffer. All cell lines were cultured at 37° C. in a humidified atmosphere consisting of 5% CO2 and 95% air. Medium was changed every 2 days and the cell morphology was evaluated under microscope daily.

Six-week-old male athymic nude mice (age 5-7 weeks) were housed with free access to food and water in a temperature-controlled room. Each group is represented by 10 xenograft cd1 nu/nu mice. The mice were injected s.c. in the right distal hind limb with one million of cultured human tumor cell lines suspended in 0.15 mL of 2:1 solution of PBS/Matrigel (Matrigel BD Biosciences). Xenografts were measured every four days with a digital caliper in two perpendicular dimensions. Tumor volume was calculated according to the following formula:

Tumor volume=(tumor width)×(tumor width)×(tumor length)÷2

At the end of experiments, mice were euthanized and tumors removed. Weight loss or other signs of distress such as hunched posture were checked during and after treatments.

Efficacy of treatment was evaluated by: (1) the size of tumors (vehicle control and treated), (2) the relative weight at the end of experiment, and (3) the indirect evaluation of angiogenesis by determining hemoglobin levels.

In vivo tumor response to treatment was evaluated using the following parameters: (i) Complete response (CR): defined as the disappearance of a measurable lesion, (ii) Partial response (PR): defined as a reduction of greater than 50% of the sum of the products of the cross-sectional diameters of the measurable lesion, (iii) Stable disease (SD): defined as a reduction of less than 50% or an increase of less than 25% in the sum of the products of the cross-sectional diameters of the measurable lesions, (iv) Tumor progression (TP): defined as an increase of greater than 50% in the sum of the products of the cross-sectional diameters of the measured lesion, (v) Time to progression (TTP) defining the tumor growth delay. Tumor progression was calculated through the analysis of bioluminescence imaging (BLI) photon counts and when possible through the analyses of tumor volumes calculated by magnetic resonance imaging (MRI), (vi) Disease free survival (DFS) is the time elapsed between the time point at which no luciferase activity is detectable anymore and the death of the animal, (vii) Overall survival (OS) is the time elapsed between tumor inoculation and death of the animal. Animals were euthanized when they displayed unethical conditions of neurological signs (e.g., altered gait, tremors/seizures, lethargy) or weight loss of 20% or greater of pre-surgical weight. Luciferase transfected cells were used for bioluminescence evaluations.

### Statistical Methods

Data are presented as mean and standard error of the mean (SEM) or standard deviation (SD). Statistical comparisons between control and treated groups were established by carrying out the Kruskal-Wallis test (a nonparametric one-way analysis of variance) or the Mann-Whitney test (in the case of two groups). The level of significance was corrected by multiplying the P value by the number of comparisons performed (n) according to Tukey's correction. TP, TTP and DFS were analyzed by Kaplan-Meier curves and Gehan's generalized Wilcoxon test. All statistical analyses were undertaken using SPSS 10.0.

### 3.2 Glioblastoma CDX U87MG

### 3.2.1 KcAng(1-7)+temozolomide

Synergy of KcAng(1-7) and standard of care, temozolomide (TMZ), against heterotopic U87MG cell culture-based xenografts (CDX) in mice.

Mice with U87MG cell CDX were randomized at day 0. KcAng(1-7) was injected daily subcutaneously (0.2 μg/kg) starting at day 0 until the (ethical) endpoint maximally at day 42. TMZ was administered at 16 mg/kg during 5 consecutive days, that is from day 2 to day 7. Volumetric evaluation of the tumor was performed every 4 days and tumor weight was measured at the end of the experiment.

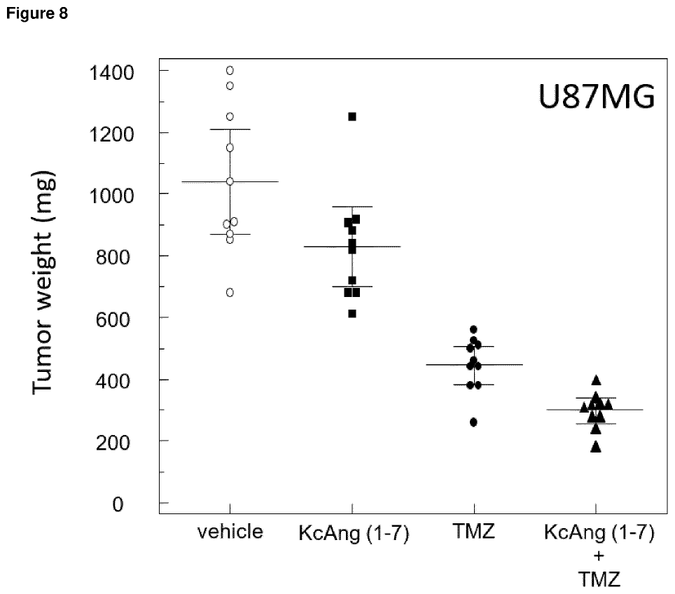

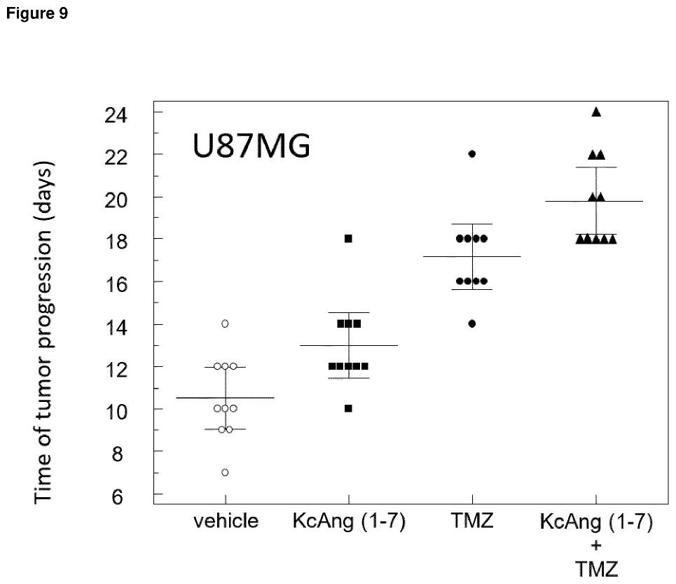

Results of tumor weight are shown in FIG. 8 and Table 10. For the combination treatment of KcAng(1-7) and temozolomide (TMZ), a synergistic effect on tumor weight loss can be shown (Table 11). Time of tumor progression is shown in FIG. 9 and Table 12.

TABLE 10
Tumor weight (mg) in U87MG CDX treated with
KcAng(1-7) (0.2 μg/kg), TMZ (16 mg/kg)
or a combination of KcAng(1-7) and TMZ.

| | mean | tumor weight | |
|---|---|---|---|
| group | (mg ± SD) | [%] | significance |
| vehicle | 1040 ± 75 | 100.0 | |
| KcAng(1-7) - | 830 ± 57 | 79.8 | P = 0.0386 vs vehicle |
| 0.2 μg/kg/d | | | |
| Temozolomide | 445 ± 28 | 42.8 | P < 0.0001 vs vehicle |
| (TMZ) | | | P < 0.0001 vs KcAng(1-7) |
| KcAng(1-7) + | 299 ± 19 | 28.8 | P < 0.0001 vs vehicle |
| TMZ | | | P < 0.0001 vs KcAng(1-7) |
| | | | P = 0.0022 vs TMZ |

Synergism was determined using the methods described in Clarke et al. Breast Cancer Research and Treatment 46:255-278 (1997).

The data is analysed in the following way:

Antagonistic: (AB)/C>(A/C)×(B/C)

Additive: (AB)/C=(A/C)×(B/C)

Synergistic: (AB)/C<(A/C)×(B/C)

Where A is response to treatment 1; B is response to treatment 2; C is response to vehicle; AB is combination of treatments A and B.

TABLE 11
Calculation of synergism using the Clarke Theorem
based on the data in FIG. 8 and Table 10.

| | Tumor weight [%] |
|---|---|
| A: KcAng(1-7) [0.2 μg/kg] | 79.8 |
| B: Temozolomide [16 mg/kg] | 42.8 |
| C: Vehicle control | 100.0 |
| AB: Combination: KcAng(1-7) + TMZ | 28.8 |
| (AB)/C [%] = observed combinatorial effect | 28.8 |
| (A/C) × (B/C) [%] = theoretical additive effect | 34.2 |
| Conclusion | synergy |

Conclusion: Since 34.2 ((A C)×(B/C)) is bigger than 28.8 ((AB)/C), according to Clarke et al., a synergistic effect of KcAng(1-7) and Temozolomide on tumor weight reduction is demonstrated. As shown in Table 12 this is also reflected in a significant (p<0.0001) prolongation of TTP in the combination treatment vs vehicle and vs KcAng(1-7) alone treatment.

TABLE 12
Time of tumor progression - TTP (days) in U87MG CDX treated
with KcAng(1-7), TMZ or a combination of KcAng(1-7) and TMZ.

| group | mean (days ± SD) | significance |
|---|---|---|
| vehicle | 10.50 ± 0.63 | |
| KcAng(1-7) - | 12.80 ± 0.54 | P = 0.0131 vs vehicle |
| 0.2 μg/kg/d | | |
| Temozolomide | 17.00 ± 0.54 | P = 0.0001 vs vehicle |
| (TMZ) | | P = 0.0009 vs KcAng(1-7) |
| KcAng(1-7) + | 20.20 ± 0.70 | P < 0.0001 vs vehicle |
| TMZ | | P < 0.0001 vs KcAng(1-7) |
| | | P = 0.0161 vs TMZ |

The subcutaneous U87MG xenograft (CDX) model revealed that KcAng(1-7) reduces the size and weight of U87MG derived tumors in KcAng(1-7) treated mice vs vehicle. KcAng(1-7) treatment alone led to a significant reduction of tumor weight. In particular, combination of KcAng(1-7) with Temozolomide led to a strong synergistic reduction of tumor mass. Furthermore, KcAng(1-7) increases the time of tumor progression by approximately 2 days leading to an increased effectiveness of temozolomide.

These results suggest that a combination of KcAng(1-7) and temozolomide synergistically inhibits the in vivo growth of U87 glioma cells.

### 3.2.2 KcAng(1-7)+Losartan

KcAng(1-7) and losartan against heterotopic U87MG cell culture-based xenografts (CDX) in mice.

Mice with U87MG cell CDX were randomized at day 0. KcAng(1-7) was injected subcutaneously (1 μg/kg) on a daily basis. Losartan was administered intraperitoneal at 20 mg/kg/day.

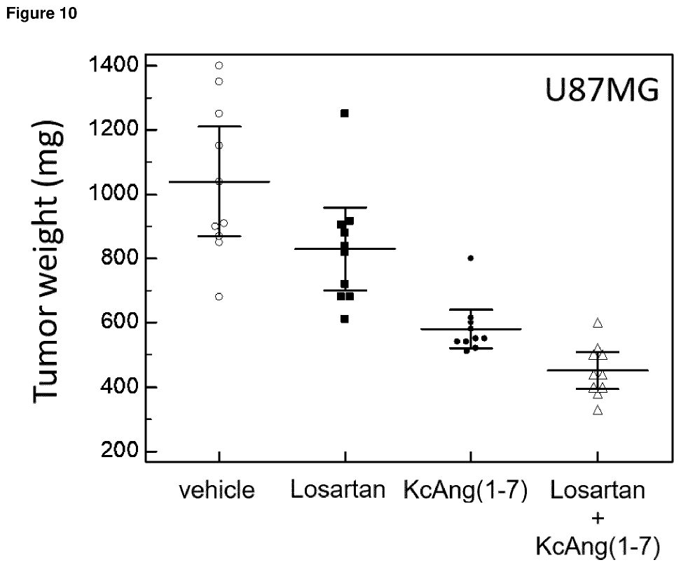

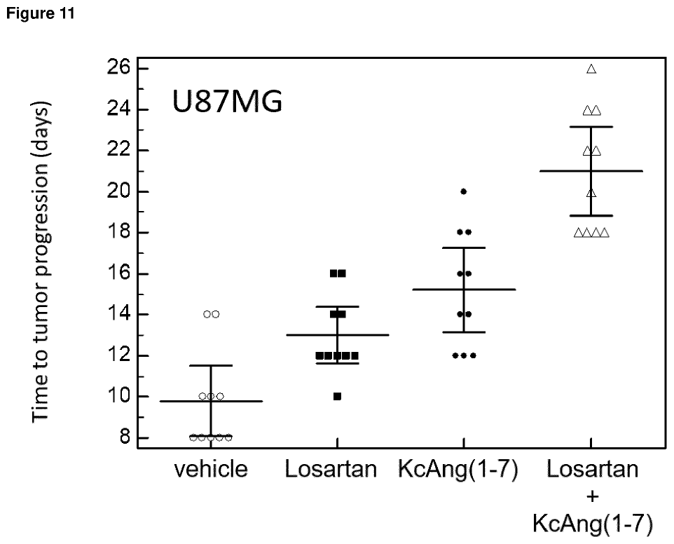

Results of tumor weight are shown in FIG. 10 and Table 12. A minor synergistic effect on tumor weight reduction has been observed in the combination group KcAng(1-7)+Losartan (Table 13). FIG. 11 and Table 14 shows the time of tumor progression.

TABLE 12
Tumor weight (mg) in U87MG CDX treated with
KcAng(1-7) (1 μg/kg), Losartan (20 mg/kg)
or a combination of KcAng(1-7) and Losartan.

| | mean | tumor weight | |
|---|---|---|---|
| group | (mg ± SEM) | [%] | significance |
| vehicle | 1040 ± 75 | 100.0 | |
| KcAng(1-7) - | 580 ± 27 | 55.8 | P = 0.0007 vs vehicle |
| 1 μg/kg/d | | | |
| Losartan | 830 ± 57 | 79.8 | P = 0.0386 vs vehicle |
| | | | P = 0.0013 vs KcAng(1-7) |
| KcAng(1-7) + | 451 ± 25 | 43.4 | P = 0.0001 vs vehicle |
| Losartan | | | P = 0.0002 vs KcAng(1-7) |
| | | | P < 0.0001 vs Losartan |

TABLE 13
Calculation of synergism using the Clarke Theorem
based on the data in FIG. 10 and Table 12.

| | Tumor weight [%] |
|---|---|
| A: KcAng(1-7) [1 μg/kg] | 55.8 |
| B: Losartan [16 mg/kg] | 79.8 |
| C: Vehicle control | 100.0 |
| AB: Combination | 43.4 |
| (AB)/C [%] = observed combinatorial effect | 43.4 |
| (A/C) × (B/C) [%] = theoretical additive effect | 44.5 |
| Conclusion | synergy |

Conclusion: Since 44.5 ((A C)×(B/C)) is bigger than 43.4((AB)/C), according to Clarke et al., a minor synergistic effect of KcAng(1-7) and Losartan may be suggested. As shown in Table 14 this also translates to a significant prolongation of TTP in the combination treatment vs vehicle, vs KcAng(1.7) and vs Losartan treatment.

TABLE 14
Time of tumor progression - TTP (days) in U87MG
CDX treated with KcAng(1-7), Losartan or a
combination of KcAng(1-7) and Losartan.

| group | mean (days ± SEM) | significance |
|---|---|---|
| vehicle | 9.8 ± 0.8 | |
| KcAng(1-7) - | 15.2 ± 0.9 | P = 0.0014 vs vehicle |
| 1 μg/kg/d | | |
| Losartan | 13.0 ± 0.6 | P = 0.0224 vs vehicle |
| | | P = 0.0484 vs KcAng(1-7) |
| KcAng(1-7) + | 21.0 ± 1.0 | P < 0.0001 vs vehicle |
| Losartan | | P = 0.0023 vs KcAng(1-7) |
| | | P = 0.0002 vs Losartan |

### 3.2.3 KcAng(1-7) Reduces Hemoglobin Content of Glioblastoma U87MG CDX

Angiogenesis was evaluated indirectly by assessing tumor hemoglobin levels (HgB) with a hemoglobin assay as described by Gravina G L, et al., Endocr Relat Cancer. 2011; 18:385-400. In brief, tumors were homogenized in double-distilled water. Eighty microliters of homogenates were mixed with 1 mL of Drabkin's solution and incubated for 15 min at room temperature. After centrifugation at 400×g for 5 min, the supernatants were subjected to absorbance measurement at 540 nm. The absorption, which is proportional to hemoglobin concentration, was divided by tumor weight.

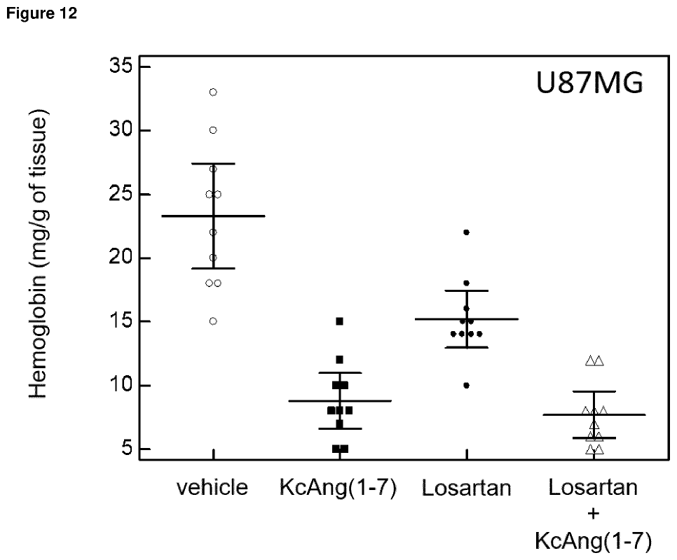

Results on tumor hemoglobin level are shown in FIG. 12 and Table 15. In all treatment groups, HgB levels were significantly decreased vs vehicle control. Lowest HgB levels were observed in the group treated with a combination of KcAng(1-7) and Losartan.

TABLE 15
Tumor hemoglobin level in U87MG CDX treated with KcAng(1-
7), Losartan or a combination of KcAng(1-7) and Losartan.

| | mean (mg hemoglobin/ | |
|---|---|---|
| group | g of tissue ± SEM) | significance |
| vehicle | 23.3 ± 1.8 | |
| KcAng(1-7) - | 8.8 ± 1.0 | P = 0.0002 vs vehicle |
| 1 μg/kg/d | | |
| Losartan | 15.2 ± 1.0 | P = 0.0025 vs vehicle |
| | | P = 0.0054 vs KcAng(1-7) |
| KcAng(1-7) + | 7.7 ± 0.8 | P < 0.0001 vs vehicle |
| Losartan | | P = 0.3517 vs KcAng(1-7) |
| | | P = 0.0004 vs Losartan |

KcAng(1-7) at 1 μg/kg exerts a stronger inhibitory effect against glioblastoma U87MG CDX than Losartan. The levels of HgB (mg/g of tissue) correlate with size of tumors and treatment effectiveness.

These data suggest that KcAng(1-7) suppresses glioblastoma growth by inhibiting angiogenesis.

### 3.3 Glioblastoma CDX U251MG

### 3.3.1 KcAng(1-7)+Losartan

Effect of KcAng(1-7) and Losartan against heterotopic U251MG cell culture-based xenografts (CDX) in mice. Animals were treated either with vehicle, treated with 20 mg/kg/d Losartan, treated with 1 μg/kg/d KcAng(1-7) or treated with a combination of KcAng(1-7) and with Losartan.

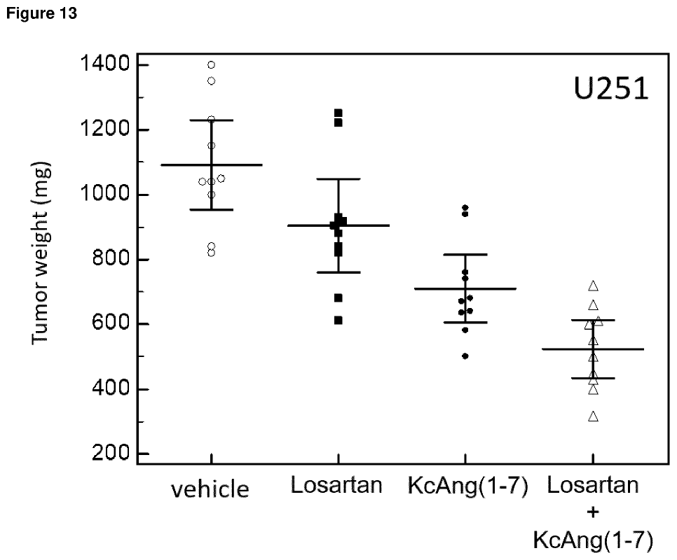

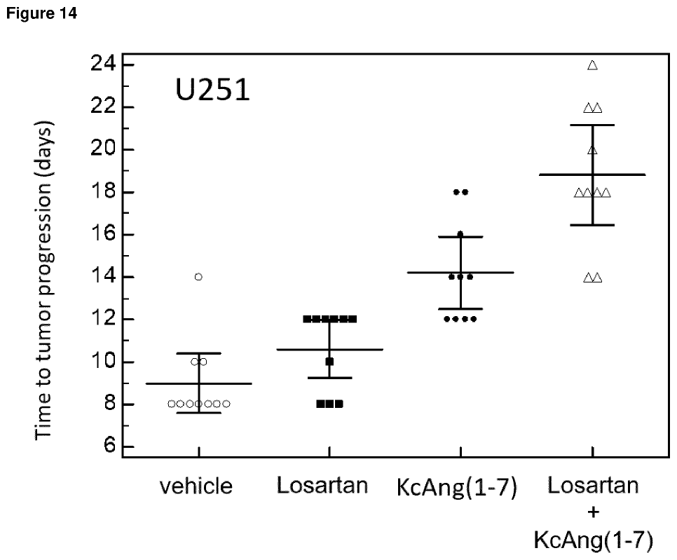

Results on tumor weight reduction are shown in FIG. 13. Time of tumor progression is shown in Table 16. KcAng(1-7) treatment alone delays tumor progression more compared to Losartan treatment alone. The strongest effect on TTP prolongation is observed in the combination treatment of KcAng(1-7) and Losartan.

TABLE 16
Time of tumor progression - TTP (days) in U251MG
CDX treated with KcAng(1-7), Losartan or a
combination of KcAng(1-7) and Losartan.

| group | mean (days ± SEM) | significance |
|---|---|---|
| vehicle | 9.0 ± 0.6 | |
| KcAng(1-7) - | 14.4 ± 0.8 | P = 0.0004 vs vehicle |
| 1 μg/kg/d | | |
| Losartan | 10.6 ± 0.6 | P = 0.1369 vs vehicle |
| | | P = 0.0120 vs KcAng(1-7) |
| KcAng(1-7) + | 18.8 ± 1.01 | P < 0.0001 vs vehicle |
| Losartan | | P = 0.0106 vs KcAng(1-7) |
| | | P = 0.0001 vs Losartan |

### 3.2.3 KcAng(1-7) Reduces Hemoglobin Content of Glioblastoma U251MG CDX

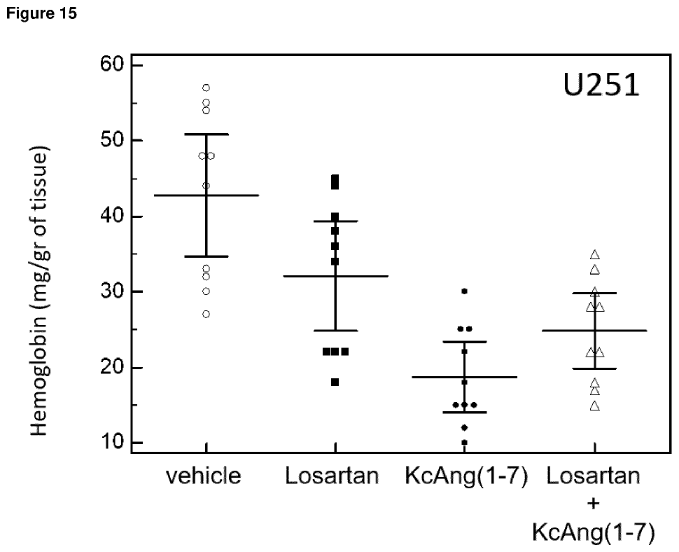

Results on tumor hemoglobin level in the U251 model are shown in FIG. 15 and Table 17.

TABLE 17
Tumor hemoglobin level in U251MG CDX treated with KcAng(1-
7), Losartan or a combination of KcAng(1-7) and Losartan.

| | mean (mg Hb/ | |
|---|---|---|
| group | g of tissue ± SEM) | significance |
| vehicle | 42.8 ± 3.6 | |
| KcAng(1-7) - | 18.7 ± 2.1 | P = 0.0004 vs vehicle |
| 1 μg/kg/d | | |
| Losartan | 32.1 ± 3.1 | P = 0.0333 vs vehicle |
| | | P = 0.0028 vs KcAng(1-7) |
| KcAng(1-7) + | 24.8 ± 2.2 | P = 0.0028 vs vehicle |
| Losartan | | P = 0.1102 vs KcAng(1-7) |
| | | P = 0.1684 vs Losartan |

In all treatment groups, HgB levels were significantly decreased vs vehicle control.

Interestingly, lowest levels of HgB were observed in the KcAng(1-7) treatment alone showing that KcAng(1-7) at 1 μg/kg exerts an inhibitory effect against glioblastoma U251 MG cell CDX.

### 3.4 Conclusions

KcAng(1-7) (0.2 μg/kg/d) administered to the in vivo U87MG CDX model in combination with the standard drug temozolomide shows a synergistic effect on tumor weight reduction.

KcAng(1-7) at 1 μg/kg exerts a stronger inhibitory effect against glioblastoma U87MG and 251 MG CDX than Losartan.

Time of tumor progression is significantly delayed in in both U87 and U251 glioblastoma CDX models in all treatment groups comprising KcAng(1-7).

Taken together, these studies suggest that KcAng(1-7) alone, or in combination with temozolomide or Losartan has anti-tumoral activity in human glioblastoma CDX models.

1. A cyclic peptide variant of angiotensin(1-7) for the use in the treatment of cancer, wherein said cyclic peptide comprises the amino acid sequence
(SEQ ID NO: 1)
Xaa1-Asp-Arg-Val-Abu/Ala-Ile-His-Abu/Ala
and a thioether-bridge between the side chains of Abu/Ala at position 5 and Abu/Ala at position 8, and
wherein Xaa1 is selected from the group consisting of Lys, Tyr, Asp, pGlu, and lle,
and
wherein said cancer is selected from the group consisting of brain cancer, colon cancer, and/or ovarian cancer.

2. A cyclic peptide for use according to claim 1, wherein Xaa1 of said cyclic peptide is a D-stereoisomer.

3. A cyclic peptide for use according to any one of the preceding claims, wherein Xaa1 of said cyclic peptide is Lys.

4. A cyclic peptide for use according to any one of the preceding claims, wherein position 5 of said peptide is a D-stereoisomer of Ala.

5. A cyclic peptide for use according to any one of the preceding claims, wherein position 8 of said peptide is an L-stereoisomer of Ala.

6. A cyclic peptide for use according to claim 5, wherein Lys is a D-stereoisomer.

7. A cyclic peptide for use according to any one of the preceding claims, wherein position 5 of said peptide is a D-stereoisomer of Ala and position 8 is an L-stereoisomer of Ala.

8. A cyclic peptide for use according to any one of the preceding claims, having an amino acid sequence of Lys-Asp-Arg-Val-Abu/Ala-Ile-His-Abu/Ala (SEQ ID NO: 2) with the provision that the peptide does not contain two Abu (2-aminobutyric acid) residues.

9. A cyclic peptide for use according to any one of the preceding claims, wherein said cancer is brain cancer.

10. A cyclic peptide for use according to claim 9, wherein said brain cancer is glioblastoma multiforme.

11. A cyclic peptide for use according to claim 9 or claim 10, wherein the cyclic peptide is administered in combination with temozolomide or with an AT1 receptor antagonist.

12. A cyclic peptide for use according to any of the preceding claims, wherein the use in the treatment of cancer comprises inhibiting angiogenesis of the tumor cells.

13. A cyclic peptide for use according to any of the preceding claims, wherein the use in the treatment of cancer comprises tumor growth inhibition.

14. A pharmaceutical composition for the use according to one of the preceding claims comprising a cyclic peptide according to one of the preceding claims and a pharmaceutically acceptable adjuvant, diluent or carrier.

15. A synergistic combination of a cyclic peptide, comprising the amino acid sequence Xaa1-Asp-Arg-Val-Abu/Ala-Ile-His-Abu/Ala (SEQ ID NO: 1) and temozolomide for use in treating brain cancer, preferably glioblastoma multiforme, wherein the cyclic peptide comprises a thioether-bridge between the side chains of Abu/Ala at position 5 and Abu/Ala at position 8, and wherein Xaa1 is selected from the group consisting of Lys, Tyr, Asp, pGlu, and lle.
